(19) United States
(12) Patent Application Publication
(10) Pub. No.: US 2002/0173033 A1
(43) Pub. Date: Nov. 21, 2002

(54) Device and method or three-dimensional spatial localization and functional interconnection of different types of cells

(76) Inventors: Kyle Hammerick, Palo Alto, CA (US); Friedrich B. Prinz, Woodside, CA (US); Robert Lane Smith, Palo Alto, CA (US); Ralph S. Greco, Stanford, CA (US); Rainer Fasching, Tiburon, CA (US)

Correspondence Address: Hana Verny, Peters, Verny, Jones & Schmitt LLP, Suite 6, 385 Sherman Avenue, Palo Alto, CA, 94306, US

(21) Appl. No.: 10/150,399

(22) Filed: May 17, 2002

Related U.S. Application Data
(60) Provisional application No. 60/291,814, filed on May 17, 2001.

(51) Int. Cl. C12M 3/00

(52) U.S. Cl. 435/305.2; 435/288.5; 435/29

(57) ABSTRACT

A device, method and process for three-dimensional spatial localization and functional interconnection of the same or different types of cells. The two or three-dimensional device comprising multiple layers containing wells for cell deposition where both the wells and layers are interconnected through microfluidic channels. A process for fabricating the three-dimensional device and a method for depositing different types of cells within the device in a functional interdependent spatial orientation thereby mimicking physiological functions. The device is useful for diagnostic assays, determination of dysfunction of certain cells in the system, quantification of production of cellular proteins, metabolites, hormones or other cellular products, for organ or tissue replacement, for co-culturing different cells, for testing pharmaceutical agents and as a bioreactor for production of biologicals.

This invention is based on and claims priority of he Provisional application Ser. No. 60/291,814 filed on May 17, 2001.

This invention has been supported by the Grant No. EIA-9618050 from the National Science Foundation and by the grant No. AR 457788 (RLS) from the National Health Institute and from VA Medical Unit. The Government may have certain rights to this invention.

### FIELD OF THE INVENTION

This invention concerns a device and a method for controlled, non-random, predetermined, three-dimensional, spatial localization and functional interconnection of different types of cells. In particular, the invention concerns a three-dimensional device comprising multiple layers containing wells for cell deposition where both the wells and layers are interconnected through microfluidic channels and wherein the different cell types are deposited in one or several layers and interconnected. The invention further concerns a process for fabricating such a device and a method for depositing different or the same types of cells within the device in a functionally interdependent spatial orientation, thereby mimicking physiological conditions that contribute to homeostasis and specialized regulatory communications between cells, tissues and organs.

The device is useful for diagnostic assays, determination of dysfunction of certain cells in the system, quantification of production of cellular proteins, metabolites, hormones or other cellular products, for organ or tissue replacement, for co-culturing different cells, for testing pharmaceutical agents and as a bioreactor for production of biological materials.

### BACKGROUND AND RELATED DISCLOSURES

Homeostasis, or a state of equilibrium, generally describes ideal conditions under which various tissues and organs function and various functions of the body are performed under influence of certain chemicals present in the bodily fluids and/or excreted by the tissues. The term describes the set of mechanisms responsible for adjustment of physiological, biochemical and genetic states. The term homeostasis encompasses such interrelations as the effect of gases, ions, chemicals, hormones, fluids on individual cells to assure their functionality. These components do not act individually and independently but are interrelated and, in true homeostatic state, function in concert and harmony with one another.

Because of the complexity of such interrelationships which often include several different types and different functions of different cells depending on each other, mimicking their cumulative function is very difficult, if not impossible, and yet cell to cell interactions are central to the function of many organs.

Moreover, many of the functional systems not only contain several different types of cells but they may be anatomically remote. Hypophysis, for example, of which different cells produce hormones controlling function of remote organs, secrets thyroxin for stimulation of thyroid gland, prolactin for stimulation of milk production, somatotrophins for stimulating growth and oxytocin affecting female reproduction organs.

Thus, it would be advantageous to have available a system, device and method which would achieve such functional interconnection and interdependence between different types of cells.

Several attempts have been made to prepare surfaces for cell culture, such as, for example, those described in ASAIO Journal, M594 (1994); J. Biomed. Mater. Res., 52:346-353 (2000); proceedings of the First Joint BMES/EMBS Conference Serving Humanity, Advancing Technology, October 13-16, Atlanta, Ga., p.851 (1999); and FASEB J., 13:1883-1900 (1999), however all of these designs afford solely two dimensional structure for cell deposition of maximum of two types of cells without providing selective interconnection through microfluidic channels.

ASAIO J., (1994), describes grooved and hole perforated surfaces suitable as microsubstrates for cell culture. In this case, the glass substrate was chemically treated to generate ablated grooves and holes for cell seeding.

BMES/EMBS Proceedings, (1999), describe a microfluidic system for culturing individual mouse embryos held stationary in a channel of the device fabricated of channel negatives on silicon wafers molded with polydimethylsiloxane bonded together. In this system, the fluid is passed over the embryo.

Journal of Biomed. Mater. Research, (2000), describes fabrication of microscopic cellular cultures which require no surface modification prior to cell seeding.

None of the three above references describes the three-dimensional microfluidic system suitable for interactive deposition of different cells or use of more than one cell type.

Attempts to co-cultivate two different types of cells is disclosed in FASEB J., (1999) which describes co-cultivation of hepatocytes with nonparenchymal cells and determination of their interaction through cellular micropatterning. Similarly to other disclosures, the FASEB Journal publication describes the method which utilizes cell deposition on two dimensional layer of maximum of two types of cells without providing selective interconnection through microfluidic channels. While the two types of cells were simultaneously co-cultured, they were co-cultured on the two dimensional layer only. No three-dimensional substrate structure or microfluidic connections were provided to permit inclusion of additional cell types and to provide the feedback of the cell-cell interaction.

It is, therefore, a primary objective of this invention to provide a device comprising a three-dimensional structure built of different layers for cell deposition, said structure interconnected through microfluidic channels providing communication between different types of cells deposited in different wells and on different layers of said device, which device allows determination of a degree of cell functionality or dysfunctionality, mimics the normal function of the cells or which device can be used as a bioreactor for replacement of cells or tissue.

All patents, patent applications and publications cited herein are hereby incorporated by reference.

### SUMMARY

One aspect of the current invention is a three-dimensional device for mimicking a two or three-dimensional spatial orientation of multiple different cell types.

Another aspect of the current invention is a process for fabrication of a three-dimensional device for mimicking a two or three-dimensional spatial orientation of multiple different cell types.

Yet another aspect of the current invention is a process for fabrication of a three-dimensional device for mimicking a two or three-dimensional spatial orientation of multiple different cell types, said process comprising construction of a multilayer structure comprising a support layer and one or more additional containment layers attached thereto wherein each containment layer comprises wells and microchannels as determined by masking layers according to a mask pattern.

Another aspect of the current invention is a mask pattern replicating and/or optimizing anatomical, histological and spatial physiological conditions.

Still another aspect of the current invention is a three-dimensional device comprising multiple containment layers interconnected with microfluidic channels permitting a controllable flow of fluid between individual layers.

Still yet another aspect of the current invention is a method for a three-dimensional spatial localization and functional regulation of different types of cells.

Yet another aspect of the current invention is a method for depositing different types of cells within a three-dimensional device of the invention in a functional interdependent spatial two or three dimensional orientation thereby mimicking anatomical, histological and spatial physiological conditions and homeostatic status quo of tissues and organs.

Still yet another aspect of the current invention is a method for diagnostic or therapeutic use of a three-dimensional device of the invention comprising the same or different types of cells deposited within the individual layers of the device.

Still yet another aspect of the current invention is a method for determination of functionality or dysfunctionality of certain cells within the system simulating a tissue or organ function, or for quantification of production of cellular metabolites, hormones, proteins or other cellular products.

Still yet another aspect of the current invention is a method for replacement of dysfunctional tissues or organs by fabricating a three-dimensional device comprising different types of cells spatially functionally deposited within the individual layers of the device wherein said layers and cells are microfluidically connected by the array of microfluidic channels, said device being suitable for implantation or functioning as bioreactor for mass production of cellular products and optionally comprising an inlet port for introducing and an outlet port for removing produced cellular metabolites, proteins, nutrient or other products.

### BRIEF DESCRIPTION OF THE DRAWINGS

FIG. 1 is a schematic for a three-dimensional device comprising three masking layers deposited on a layer of substrate, each layer consisting of one to several wells for cell deposition wherein said layers are interconnected with each other by microfluidic channels.

FIG. 2 is a top and side view of the device surrounded by the support layer. FIG. 2A is the side view of the support layer of the device. FIG. 2B is a top cross-sectional view of the device.

FIG. 3 is a diagram showing wells of different sizes and slopes interconnected with microfluidic channels.

FIG. 4 is a flow chart for the process for direct fabrication of silicon containment layers using masking concept.

FIG. 5 is a flow chart illustrating indirect fabrication of a mask layer from a design of containment layer.

FIG. 6 is a cross-side view of three-dimensional containment structure with cavities.

FIG. 7 shows stained chondrocytes deposited in PDMS wells. FIG. 7A shows emplacement of one cell in a round well and one cell in a square well (FIG. 7B).

FIG. 8 shows chondrocytes emplaced within 75 Am (FIG. 8A), 100 μm (FIG. 8B), 125 μm (FIG. 8C), 150 μm (FIG. 8D), 175 μm (FIG. 8E) and 200 μm (FIG. 8F) wells, stained for glycosaminoglycan-rich extracellular matrix by Safranin-O, respectively.

FIG. 9 shows an optical image of polydimethylsiloxane (PDMS) layer on a planar substrate creating longitudinal (FIG. 8A) and square well structure.

FIG. 10 is a containment layer seeded with cells.

FIG. 11A represents three-dimensional substrate seeded with three types of cells, namely, hepatocytes, fibroblasts and chondrocytes and FIG. 11B is high power image of the same FIG. 11A.

FIG. 12 is a gel representing results of the albumin production by hepatocytes cells seeded within the three-dimensional device together with fibroblasts. Lane (a) represents fibroblasts only, lane (b) represents hepatocytes and fibroblasts and lane (c) represents hepatocytes only.

### DEFINITIONS

As used herein:

“Cell” means and includes an eukaryotic cell, prokaryotic cell, yeast and, particularly, a mammalian cell.

“Surface modification” means physical or chemical modification on the surface of a containment layer and includes but is not limited to a formation of channels, canals, ducts, conduits, tubes, masking techniques, plasma surface treatments, biological coating using polysaccharides, proteins, peptides, polymers, or glycoproteins, covalent linkages of peptides, integrins, nucleic acid, saccharides, lipopolysaccharides, or amino acids, micromachining, wet or dry lithographic etching, plasma fluorine-based plasma etching, reactive ion etching, sputtering, plating, chemical and vapor deposition, physical vapor deposition and photoresist.

“Well” means a selectively designed topological feature on the surface of the containment layer in the nanometer, micrometer or millimeter range. The well may be of any size, depending on whether one cell, several cells, cluster of cells or piece of tissue is deposited there. The well may be of any shape, such as circular, square, triangular, oval or any depth, such as it may be deep (several millimeters) or shallow (0.1 micron) or it may be only an indent.

“Microfluidic channels” means a structure, such as a connection, canal, conduit, duct, or tube, where individual channels of about 1-200 micron size are connected with wells and/or interconnected with each other, permitting a flow of fluid into and from the well, where such flow is controllable and where such fluid performs certain autocrine or paracrine functions or provides mechanical stimulation in a circuit-like fashion. There may be several independent microfluidic channel systems present in one device.

“Interconnecting fluid” means a medium, water, saline, buffer, biological fluid or any other physiologically acceptable fluid or solution. There may be several independent fluids present in microfluidic channels of one device.

“Support layer” means a supporting containment layer or surrounding structure comprising inlet or outlet ports or gates permitting selective inflow and outflow of fluid(s). The support layer may be a first or a bottom layer within the three-dimensional structure and may form side and upper supporting and enclosing structured layers, or both.

“Anastomose” or “inlet port” or “outlet port” means straight or branching inlets, outlets and gates connecting the device of the invention with the external environment through which nutrition, interconnecting fluid, medium, biological or other fluids are supplied to the internal space of the device and through which metabolites or cell products are removed from the device.

“Mask pattern” or “masking pattern” means a design generated to replicate and optimize physiological conditions of the organ or tissue to be reproduced. Typically, the mask pattern is computer generated.

“Mask” means and is created from a mask pattern and is a physical replication of the mask pattern. The mask is typically generated by chrome etching of the glass layer pattern determining the structure of a masking layer. The mask embodies instruction set for a pattern directly corresponding to a pattern, or a portion of such pattern, of the layer positioned directly beneath it. The mask represents optimization pattern for deposition of cells of different sizes and for channel interconnection for each specific device. The mask facilitates and permits directed deposition of cells into the wells of the layer positioned directly beneath it and is fabricated of the material which allows cells to transfer or penetrate into and/or be deposited in the wells preferably preconditioned with selective cell adhesion molecules, such as fibronectin, vitronectin, growth factor, poly-D-lysine, etc. Upon such cell deposition, the mask itself may be peeled off removing the remnant of the cells which did not penetrate into the wells.

“Masking layer” means a non-disposable or disposable sacrificial layer which permits, assists in or enhances depositing either a layer of certain structural material for building of the device of the invention on the layer positioned directly beneath it, or a peel of layer prepared by the deposition of a mask on another layer imprinted with certain masking pattern corresponding to a pattern of the layer positioned directly beneath or directly above it and wherein, after depositing the masking layer, the mask layer could be removed, preferably by peeling-off or lifting, or can remain in place.

“Wafer(s)” means a rigid, usually round object on the order of 50 to 700 microns thick and ranging from 3 to 8 inches in diameter composed of single material, such as crystalline silicon, quartz, Pyrex, glass, silicon composed of single crystalline or epitaxial grown silicon of a different composition. The wafer may be made of any other materials known now or which will become known in the future. The wafer may be cut into size and shape of the device and typically serves as a starting material for fabrication of the containment layer or support layer.

“Containment layer” means a layer within a three-dimensional structure which contains a multiplicity of wells occupied by the same or multiple different cell types connected with microfluidic channels containing an interconnecting fluid. Containment layer may be a wafer.

“Array” or “pattern” means a defined configuration of cells emplaced within the wells and microfluidic channels relative to each other based on distance or depth of X, Y, Z axes wherein X axis is horizontal, Y axis is vertical and Z axis is inclined (slanted) to a variable degree.

“Covalent linkage” means linkage of two compounds through a pair of atoms of which each atom contributes one electron to form a pair of electrons.

“Biological coating” means a coating using biological material or coating of biological material with, for example, polymers, alginates, fibronectin, growth factor, polysaccharides, and other biological materials.

“PDMS” means polydimethylsiloxane.

### DETAILED DESCRIPTION OF THE INVENTION

The invention described herein provides a three-dimensional system which permits mimicking of the physiological interaction of mammalian cells organized within tissue or organs. The system is based on a three-dimensional device comprising multiple layers, each layer comprising a pattern of multiple wells allowing emplacement of different types of cells on the different layers, or in different wells of the same layers, or both. The layers and wells are interconnected by microfluidic channels permitting flow of product of cellular metabolism or secretion and/or flow of nutrients between the individual layers and individual wells.

This three dimensional arrangement allows simulation of actual function of tissues and/or organs, determination of functionality or dysfunctionality of individual cells within the system and/or quantification of their functionality.

The invention thus concerns a development and fabrication of three-dimensional devices comprising an array of wells and microfluidic channels, a method for depositing different types of cells within said wells, a process for fabrication of said device, a method for development of anatomical, histological and spatial orientation of different types of cells mimicking the actual physiological state of the tissue or organ and a method for assessing of function of different types of cells by detection, determination, measurement and quantification of metabolites, hormones or other products produced or secreted by the different cells and cellular interaction through actions of these products. The invention further concerns a three-dimensional device serving as a bioreactor for production of biological materials or as a transplantable unit.

I. Three-Dimensional Device

The design of the three-dimensional device of the invention takes into consideration anatomical, histological and spatial organization of complex organs and tissues which are composed of individual differentiated cell types that react within a localized environment to influence the action(s) and the feedback(s) of the surrounding cell types. The device is, therefore, designed to provide a multilayered structure wherein arrays of cells emplaced in wells and interconnected with microfluidic channels recapitulate the function of a functional system. Consequently, a design of the individual devices differ from each other depending on the tissue or organ to be replaced or of which the function is to be determined, studied or mimicked.

Utility of the device and the cellular systems where the device is used is described below.

A. Fabrication of a Three-Dimensional Device

Fabrication of three dimensional devices with organized regions of different types of cells requires nano or microfabrication of containment layers for individual cells, groups of cells, cluster of cells or pieces of tissue or organs.

The basic process involves, as a first step, a design of a masking pattern for each individual containment layer and of each individual device. As a second step, the individual support and containment layers are fabricated. In the next step, the cells are selectively deposited into the wells using masking layer technology according to the invention under sterile conditions. In the last step, the multiple layers are assembled into the device.

Masking technology involves a method for physical or chemical defining of regions within the device where cells are required, permitted or prohibited. Masking technology also includes masking steps permitting deposition and placement of different cell types within a two dimensional containment layer subsequently built into the three-dimensional device.

The device typically contains multiplicity of the two dimensional containment layers with emplaced cells, said layers requiring positioning of the cells relative to each layer to direct intracellular communication. Two or more types of cells may be positioned within one layer or in alternative, each layer may contain only one type of cell. Each layer containing the same or different type of cells is typically prepared individually.

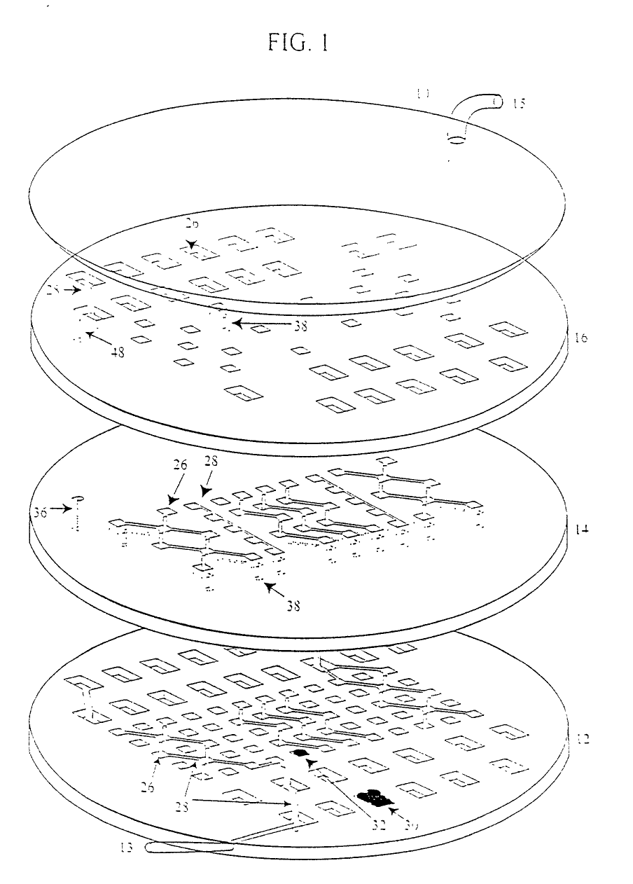

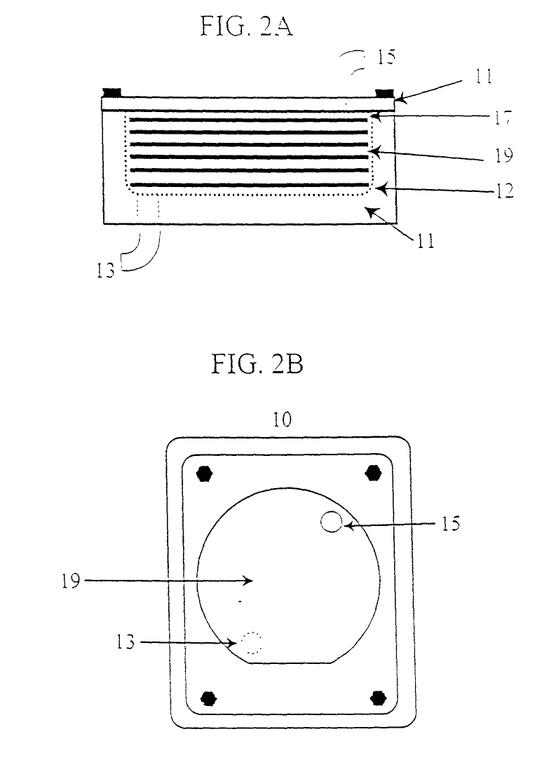

Containment layers containing cells are then deposited in organizational pattern resembling and/or reproducing the functional tissue or organ and combined into a three-dimensional structure, as seen in FIG. 1.

1. Design of the Three-Dimensional Device

A process for fabrication of the device of the invention permits designing a three-dimensional structure for three-dimensional localization and organization of the same or different types of cells, depending on the expected utility of the device.

In accordance with tissue or organs to be replaced, replicated or mimicked, the cells are organized in the same or similar fashion as in in vivo functioning organ or tissue. The device is, therefore, inherently designed to be a functional replica of the tissue or organ of which cells are investigated. In the case of a simple bioreactor, the cells are organized for maximum optimal production of the product. In the case of implant, the device may be minimized and is, typically, designed for long term functioning. The implantable device is made of biocompatible material and when the implant is designed as temporary replacement, it may be made of biodegradable material.

The actual device may or may not resemble the actual tissue or organ, however, it does function in organizational manner as such tissue or organ. The three-dimensional design of the device thus concerns more the function than the shape of the tissue or organ.

This three-dimensional design surpasses the limited existing structures disclosed previously which are designed for two dimensional co-culture of up to only two types of cells or three dimensional cultures of only one cell type without providing any interactive interconnective channels.

The current invention is designed to result in fabrication of fully functioning devices resembling organs and tissues that require specific cell organization in order to mimic the normal physiological function of the replicated organ.

2. Fabrication of the Three-Dimensional Device

Fabrication of the three-dimensional device of the invention comprises microfabrication and nanofabrication process. These processes typically consist of wafer processing steps to lithographically create containment layers with wells and microfluidic channels for intercellular communication and cellular products processing.

a. Mask Pattern

A design of the mask pattern depends on and takes into consideration anatomical, histological and spatial organization of desired tissue or organ replication.

The mask design depends on a shape, type and orientation of the cell, such as in case of gland cells which are oriented into two polarities where one polarity exhibits specified cell structures such as microvilli for secretion of the cellular product and the basal end provides signaling for the organization of the intercellular components.

The mask for each layer is designed individually for two dimensional emplacement of the cells. Consideration is given to intended cell communication with cells emplaced in other layers.

Size and shape of individual cells determines the physical aspect of the well. For example, for deposition of chondrocytes, where the cell size is typically about 25 μm and exhibits rounded morphology, the well is designed to accommodate and maintain cell size and shape of at least 25 μm. Microfluidic channels are designed to have dimensions which would accommodate flow of the fluid between layers and cells.

In one embodiment, the design of the mask pattern preparation utilizes a computed design program, such as L-Edit from Tanner Data Base Research, or any other geographical computer program.

b. Substrates for Fabrication of Support and Containment Layers

The list for substrates suitable for fabrication of containment and support layer includes the following biocompatible materials: rigid nonbiodegradable wafer materials, glass, Pyrex, quartz, silicon, polydimethylsiloxane and other nonbiodegradable biocompatible silicone based polymers and other nonbiodegradable biocompatible materials such as various metals and polymers; biodegradable polymers such as polylactic acid (PLA), polyglycolic acid (PGA), copolymer of both poly (lactic-co-glycolic) acid (PLGA), poly-β-hydroxybutyrate (PHB) polyanhydrides, polyorthoesters, polycaprolactones, polycarbonates, polyfumarates and polymethylmethacrylate; biodegradable hydrogels such as Type II collagen-GAG (glycosaminoglycan) copolymer, Type I collagen-κ elastin, alginate, calcium alginate, hyaluronan, methacrylated variations of alginates and hyaluronan and agarose. Other materials which have similar properties and are known now or will become known later are also intended to be within the scope of this invention.

c. Support Layers

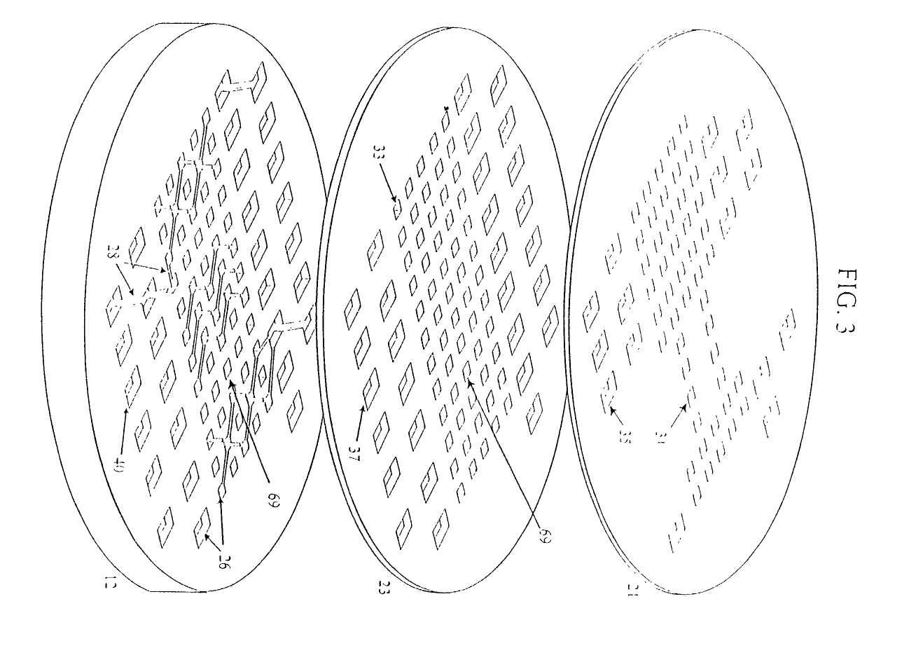

A support layer is a supporting and/or other layers surrounding structure comprising gated or nongated inlet or outlet ports or gates permitting selective inflow and outflow of fluids and cellular products. The support layer has a shape of an enclosure, as seen in FIG. 2, may be the top and bottom layer of the three-dimensional structure and may form side and other supporting structures. Support layer thus defines an external boundary of the device and forms a structural protection to the containment layers.

Within the device, individual layers are attached to each other starting with the support layer which is typically made of the different, harder and more sturdy material. The support layer may or may not contain the wells and microfluidic channels. If it does contain the wells and microfluidic channels, these are patterned into the layer using techniques such as lithographical patterning, mechanical or chemical etching, grafting, laser or machining, as described above.

The device of said support layer is flat or have a rising edge or a rim to form a containment structure comprising a cavity for emplacement of one or several containment layers within said cavity.

The support layer is typically a piece of flat material such as glass, Pyrex, quartz, polydimethylsiloxane, biocompatible silicone based polymer, biocompatible metal or polymer, silicon, hardened polymer, titanium, porous material, film, which may have a smooth surface or etched surface to facilitate assembly and stability of the device. Other materials listed in section (b) are also suitable. Typically, the support layer will contain one or more inlet or outlet ports or gates which may be straight or branched with the support layer, containment layers or within the device.

The ports and gates dictate the directional mass transport of nutrients, metabolites, cellular and biological products, fluids, etc.

In one example, the support layer is a commercially available wafer which is micromachined to a desired shape and inlet and outlet ports are attached, for example, by drilling hole(s) and attaching tubing.

d. Containment Layer

The three-dimensional device comprises of at least two but preferably three or more, up to about 1000, containment layers. In certain embodiments, it may consist of thousand or several thousands of layers, for example where the device structurally as well as functionally resembles and replaces the whole organ, such as, for example, pancreas, adrenal gland, thyroid or liver lobe or where the device is used as a bioreactor for production of biological material.

The layers may be of the same or different size or shape and may be made of the same or different materials such as those listed in section (b). The preferred materials are, for example, rigid nonbiodegradable wafer materials, glass, Pyrex, quartz, silicon, polydimethylsiloxane, thermoplastics, nonbiodegradable biocompatible silicone based polymers, nonbiodegradable biocompatible metals and polymers and other materials.

The support layer may additionally have rising edges or rim to form a containment structure comprising a cavity for containment of the second and subsequent layers within thus formed cavity. This feature permits the three dimensional structure located within such a cavity to function in a completely enclosed environment similar to the tissue or organ. Such containment structure may additionally have one or several conduits connecting an interior organization with the exterior of the containment structure. Such conduits serve as inlet and outlet ports for introducing medium or other fluid into the device and for removing products of cellular metabolism and excretion.

The containment structure described above additionally serves as a stand alone bioreactor or microfactory for production of biologicals, as a replacement for temporarily or permanently dysfunctional organs or tissue.

e. Fabrication of Containment and Masking Layers

The current method for fabrication of three-dimensional device and particularly its layers is similar to standard semiconductor device fabrication techniques and employs lithography or other micromachining methods to generate patterned wafers.

Using this techniques, containment and masking layers of the invention which consists of lithographically patterned wafers are etched to create specifically designed structures. These structures can be transferred in the negative to another material such as, for example, an elastomeric material polydimethylsiloxane (PDMS). This material can be utilized in a similar fashion to the masking wafers described below, creating a physical lift-off mask to generate patterns of cells within the containment structures.

The individual containment or masking layers can be chemically modified to make them more or less adhesive to one another using chemical polymers, such as polyethylene glycols of various molecular weight and charge, or its high molecular weight alternative polyethylene oxide, or by chemisorbing alkylsilanes and fluoroalkylsilanes. This permits the creation of reversibly adhesive layers as masks and irreversibly adhesive layers of containment structures as tissue layers. The layers may also be plasma treated to selectively transform surfaces from hydrophobic to hydrophilic.

Additionally, the layers can be surface altered chemically to create regions of cell adhesivity using, for example, the extracellular matrix macromolecules, such as fibronectin, vitronectin or peptide containing specific cell recognition sequences in the form of amino acid, or to create regions of cell repulsiveness using metallic ions, charge density or surface energy properties to exclude selective cellular responses.

Masks and containment structures for the device or bioreactor may be manufactured from a variety of materials, in addition to silicon. In particular, biodegradable and biocompatible materials permit application for usage of bioreactors and the device as medical implants.

Three-dimensional containment structures described herein can be used as implantable devices. For example, one such implantable device would be an islet cell device in which islet cells, including the component cells of the pancreatic islets and the matrix proteins are placed in the three-dimensional device of the invention. A liver device containing hepatocytes, fibroblasts and matrix proteins is suitable as a permanent or temporary relief or as a functioning unit to bridge the interval before liver transplantation. Similar devices containing neural networks, endocrine cells, etc., may be developed to treat neurodegenerative disorders, hypoparthyroidism and pain. T h e implantable device which is manufactured from biodegradable material may dissolve over time, leaving only the embedded cells behind.

Biodegradable materials include, for example, polylactic or polyglycolic acid. Examples of the biocompatible materials include hydroxyapatite and glass ceramics. These materials may be shaped into three-dimensional cavities or microchannels in different ways. Dry and wet etching processes permit creation of shape features with micrometer dimensions directly from the biocompatible or biodegradable materials. Alternatively, a silicon master shape may be transferred into biodegradable or biocompatible materials with the help of a sacrificial mold. For such a case, the master material is removed from the mold by dissolving or sacrificing the silicon master. A number of agents, such as, for example, potassium hydroxide, are available to dissolve silicon. Next, the cavity is filled by injection molding, casting or gel-casting with the biodegradable or biocompatible material. Finally, the mold is sacrificed. Cells are then embedded into the biodegradable or biocompatible materials in a fashion similar to described above and below.

f. Lithographic Process for Fabrication of Containment Layer

A typical lithographic process may consist of signing the wafers at 100-200° C., preferably at 150° C. for at least 30-60 minutes to dehydrate the surface and drive off unwanted residual water. Surface is treated with hexadimethylsilane (HMDS) to promote adhesion of the resist coating. A photoreactive polymer more commonly known as photoresist, is applied to the wafer surface by spin coating the wafer with the resist. Resists which were used in this process are Shipley AZ3612 resist spun at thicknesses of 1 to 1.6 μm and Shipley SPR220-7 commonly spun to thickness of 7 μm. The resist coated wafers are postbaked to remove solvents from the resist coat.

The resist coated wafers are then placed in a contact aligner (Karl Suss MA-6 Contact Mask Aligner or Electronic Visions 620 Aligner). Near UV light is used to illuminate a chrome on glass mask to expose the wafers. Typical exposure energies are 28.7 mCal/cm² for SPR220-7 7 μm resist and 1.6 μm 4.87 mCal/cm² for AZ3612 resist. The positive resist is solubilized by the UV light (17 mW/cm²) where it reaches the polymer surface.

The wafers are developed in developing solutions Shipley MF-319 or LDD-26W for various times depending on the resist type and thickness. The wafers now have a layer of patterned polymer over bare silicon. The exposed silicon can be dry etched away to create topological features.

g. Masking Layers and Process for Cell Deposition

A process according to the invention comprises selective depositing of the cells and/or layers withing the three-dimensional device according to a specific mask pattern. Mask patterns are predetermined and contain numerous cavities forming wells and tubular conduits forming microfluidic channel connection between the cells and layers.

i. Fabrication of Masking Layer

The mask is processed in a similar fashion as described above in sections (e) and (f) with the etching creating openings, such as holes, for cells to pass through and into the wells positioned the containment layer immediately below. The pattern of the holes etched on the masking layer, such as the wafer, corresponds directly to the pattern of the etched wells of the bellow containment layer.

ii. Application of Masking Layer

Typically, for this step, the mask or wafer is affixed to the substrate layer or to the layer positioned directly below the mask and cells can be transferred from the mask to the wells of the lower layer by centrifugation through the mask holes, or using pressure, electrophoretic deposition, electrophoresis, electrical current or vacuum. The material on which the cells were originally deposited may then be peeled-off. The peeled-off material removes the remainder of the cells which were not transferred into the wells of the lower layer.

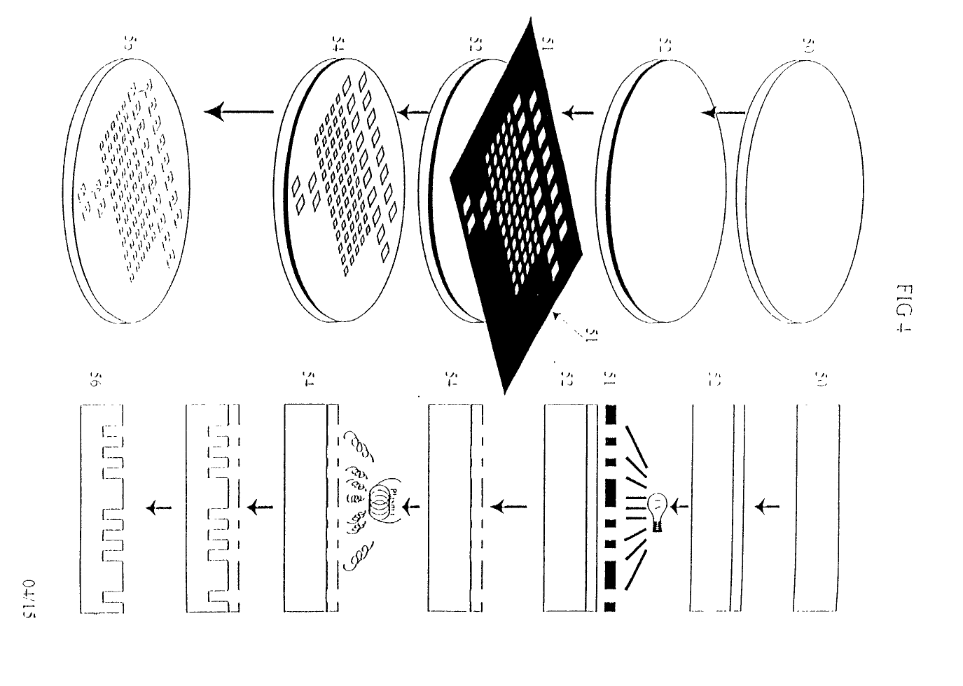

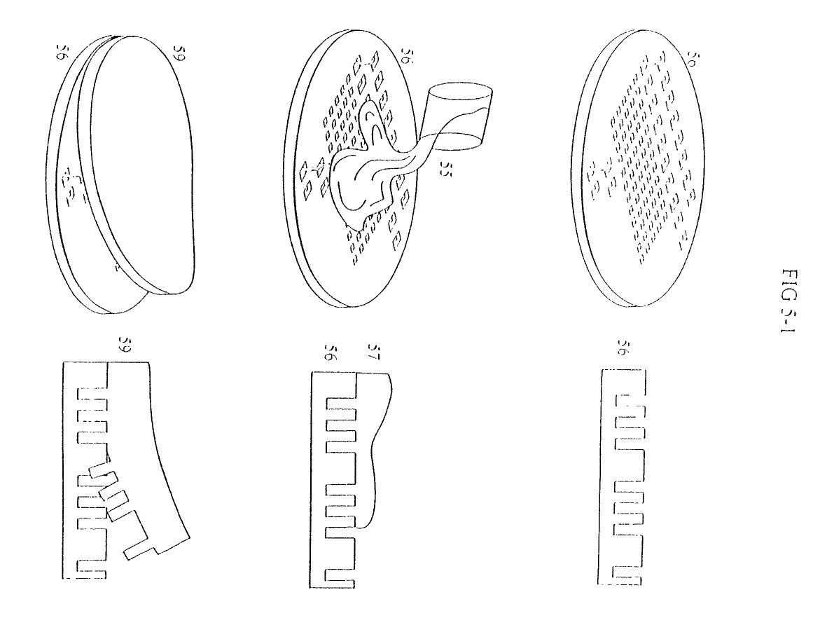

This step may be repeated several times with the masking layer comprising a different patterns of holes for deposition of different types of cells, thereby permitting fabrication of the layer comprising more than one type of cell. By applying various masking layers to the containment layers, specific and definite patterns of different cells within the device layers can be attained, as seen in FIG. 3. The containment layers can then be placed one on the top of another and combined to create three dimensional structures of cells, as seen in FIG. 4.

Typically the process for cell deposition comprises fabrication of a mask and masking layer and a masking wafer. The mask or masking layer, in one embodiment, consists of a layer of an adhesive or other material to which the layer of cells is attached, for example by spraying, spray-drying, adhesion, chemical deposition, attraction, etc.

3. Fabrication Process Steps

A fabrication methodology for building the above device comprises the following steps:

First, the internal three-dimensional structure of the device is designed according to the intended purposes depending on the tissue or organ to be replaced or mimicked.

The design pattern of the three-dimensional device comprises the outer design of the support layer(s) and the inlet and outlet ports, internal design of the containment layers including wells and microfluidic channels for intercellular flow connections. The ratio of wells, microfluidic channels and other cavities is controlled to optimize the interactive conditions and cell adhesion.

Second, the individual layers of the device are fabricated and, as the last step, emplaced within a support layer.

The external support layer surrounds the internal cavity of the device and is made of harder material such as quartz, glass, or silicon.

A design of the distribution of wells and microfluidic channels is then introduced into containment layers according to the mask pattern. Such design takes in consideration an actual structural orientation of each cell type to another cell type.

The wells have dimensions from about 1 μm to about 200 Am, preferably from about 25 μm for single cell deposition, to about 75 μm deposition for deposition of several cells. For deposition of multiple cells, cell clusters or tissue, the wells are in the millimeter range. Some or all wells are interconnected within the containment layer by microfluidic channel network. The wells thus may be connected with upper layers via microfluidic channels horizontally, vertically or inclined (slanted) sideways. There may be one or several independent microfluidic channel networks connecting wells.

Microfluidic channels are the canals, conduits or tubes of about 1 to about 200 μm diameter connecting the wells positioned on different containment layers. Microfluidic channels permit the flow of cellular products, such as metabolites, hormones, proteins, peptides, etc., and/or nutrients supporting cell function, to or from the cells placed in the wells and additionally provide connections between the individual wells or individual layers.

Certain variations of the layer preparation include depositing cell adhesive proteins onto a containment layer or into the wells to provide adhesion for the second layer or adhesion for cells to the walls of the wells.

Third, the cells are seeded into the wells of each individual containment layer. This step is simultaneous with the second step or follows the second step depending on the technique used for seeding.

In the process of seeding the cells, cells are deposited directly on each layer, starting with the first containment layer. The first containment layer is then covered with the next containment layer having already deposited the same or different cells in its wells using masking layers, as described above. The second layer is covered with the third, fourth, fifth, etc., containment layer having deposited cells in the wells, as described. When the whole three-dimensional device is built and enclosed within the support layer, the medium or other fluid is introduced into microfluidic channels, for example, by injection, by submerging the device in the medium, by perfusion, micropump, osmotic gradients, electrical current, vacuum, or by diffusion, etc.

Cells may be deposited in the wells directly or by injecting the appropriate microfluidic channel with a cell suspension for example, under pressure, by centrifugation, by suction, spraying, inkjet printing or spin-on in a centrifuge.

For the purpose of selectively depositing different cells into different wells, lithographically prepared masking layers allow for the deposition of one kind of cell into selected array elements. Excess cells may be removed by masking layer lift-off or peel-off.

Cells are seeded onto individual layers before these layers are mounted together and they are deposited using masking layers as described above.

Additionally, the process can be repeated by depositing different cells into different containment layers by applying a second set of masking layers followed by lift off.

Fourth, the layers are mounted together to form the three-dimensional structure. The three-dimensional structure is prepared by incremental build-up of individual containment layers. Layers are typically held together by mechanical restrain such as clamp or chemically bonded to each other using adhesives, proteins, glycoproteins, glues, wax, silicon grease, etc.

Layers are aligned, optically by physical alignment or by mask contact alignment, to be held in a specific predetermined orientation within error ±1-3 μm.

B. Detailed Description of Drawings

The device of the invention, its design and method for its use is illustrated in FIGS. 1-12.

FIG. 1 shows the device of the invention wherein the spatial organization as well as microfluidic interconnection between the individual microwells of each layer as well as between individual layers is graphically depicted.

As seen in FIG. 1, the device 10, in this particular instance, comprises of four layers 12, 14, 16 and 18. The layer 12 is the first containment layer, typically made of material such as glass, polymer, silicon polydimethylsiloxane, biopolymers, etc., or other materials described above. In the alternative, the layer 12 could also be a support layer serving in the same way as the containment layer. This figure shows the containment layer 12 as containing wells 26 and microfluidic channels 28. In another arrangement, the support layer 12 does not contain wells with cells, but contains conduits for bringing and removing fluids or, in alternative, it contains both.

Layer 12 contains an array of microwells 26 of sizes typically between about 1-100 μm suitable for holding one or more cells, cell clusters or pieces of tissue. Preferably, only one cell or a cell cluster is deposited per well. The microwells are or are not interconnected with each other, depending on the design of the device or, in the alternative, only some may be connected with each other within the layer by microfluidic channels 28. The microwell 30 is shown holding several cells, i.e. a cluster of cells. Microwell 32 is shown holding one cell.

When the containment layer 12 is generated, cells of the system which are intended to be placed there are deposited into the microwells by using, for example, centrifuging, infusion, submersion, pressure injection, electrical current, electrical potentials (electrophoretic and dielectrophoretic methods) etc., as described above, or adhesion and/or repulsiveness, using appropriate adhesive or repulsive materials, may also be used to attract deposition of cells in only some but not all wells.

The containment layer 12 is prepared as shown in FIG. 4 and then covered with the second layer prepared with cells in a similar manner, such as for example, the containment or masking layer 14 which also contains wells 26 interconnected with microfluidic channels 28 within the layer 14 and also connected with layer 12 through microfluidic channels 38 connecting the wells of layer 14 to the wells of layer 12.

The masking layer 14 is deposited on the layer 12 and attached, for example, by chemically induced adhesion or mechanically applied pressure, to the layer 12. The same or different types of cells are deposited in microwells 26 of the layer 14 than those cells deposited in microwells of layer 12.

Layers 14 and 16 and additional layers which are not shown in FIG. 1 but may be added to the three layers shown herein are prepared essentially as described for layer 12. They contain wells 26 for deposition of one or more types of cells and microfluidic channels 28 interconnecting microwells 26 among themselves. Some of all microwells are also connected with other remote containment layers, namely they may be connected only with layer 12, with layer 14 or with both, as well as with additionally added layers.

The microfluidic channels may connect directly, for example, layer 12 to layer 16 channel, or to any additionally added layers as illustrated, for example, by the microwell 26 of the layer 12 which is connected to the microwell 26 of the layer 14 which, in turn, is connected in this instance to the microwell 26 of the layer 18.

In this regard, it is preferred that the individual layers are either hard layers made of the hard material, such as glass, quartz, silicon, silicon oxide, polymers, diamonds, metals, etc., or are the combination of so called hard layers and soft layers made of the soft material, such as collagen, polylactic acid, polyglycolic acid, poly(lacticglycolic) acid, polydimethylsiloxane, polymethylmethacrylate, or other biodegradable or nonbiodegradable polymers. This arrangement provides several advantages. The hard layer, or the hard mask, permits an exact alignment of microfluidic channels with precision of about one micron accuracy, permits fabrication of the layers and masks with precise sizes and shapes of the wells and microfluidic channels and connections between the wells and layers and permit the easier registration of the cell or cells clusters emplacement. In one embodiment, the hard layers may be augmented with soft layers, preferably interspaced with one soft layer between two or more hard layers. Soft layers provide of advantage of isolating two hard layers and easy removal of the soft layer by peeling. This feature may be advantageously utilized for seeding of several types of cells on one layer by applying two or more soft peelable layers in sequence each permitting seeding of one type of cells in predetermined wells on the containment layer. Although possible, the use of only soft layers is not very advantageous for the current invention as these layers easily deform and thus lack ability of the precise formation of wells and two or more soft layers are also difficult to precisely align.

Versatility of the device design is apparent from the above description as any possible arrangement is intended to be within the scope of this invention.

Connection of microfluidic channels through the several layers is achieved by directing these microchannels to non-neighboring remote layer through troughs 36 as seen, for example, as microfluidic channel 48 which is directly connected to layer 12 with containment layer 12 through troughs 36 on layer 14.

FIG. 2 is a top and side view of an example construction of the device 10. The support layer which forms a cover for the device 10 and isolates the device interior space from the exterior space surrounding the device is shown in FIG. 2 as layer 11. The support layer has an inlet port 15 and an outlet port 13 shown in detail in FIG. 2A. The support layer is typically a structure made of solid material with no holes, conduit, microchannels, troughs or wells but may be manufactured from porous material. Its function is to cover the 3-dimensional structure.

FIG. 2A shows the side of the device wherein the shape of the device 10 is determined by the support layer 12. The support layer 11 provides connection between the internal space of the device 10 and external space by inlet port 15 and outlet port 13. Inlet and outlet ports may be located on top and bottom of the device, as seen in FIG. 2A, but may also be inverted, that is, the inlet may be located on the bottom or on the side, and in any combination thereof.

The support or cover layer 11 has a supporting function as encasing the layers 12-18 (or any additional layers as may be added) within the definite structure separated from the external environment.

The inlet port 15 and outlet port 13 are typically round tubes attached to the support layer or molded as a continuation of the support layer or are directly a part of the support layer. These ports are used to add or remove fluid from the internal space 19 of the device or may be used to deposit the cells within the layers. FIGS. 2A and 2B show a simplest design of the inlet and outlet ports. It is, however, to be understood that these ports are connected either directly with microfluidic channels 28 or from a branched or straight circulature within the internal space of the design which ultimately connects the inlet port with the outlet port.

The outlet port is used to remove fluid containing metabolites and cellular products which are then subjected to biochemical, histological or other laboratory tests.

FIG. 2B is a top view of the device 10 showing a placement of a tubular inlet port 15 and outlet port 13 connecting internal space 19 with external environment.

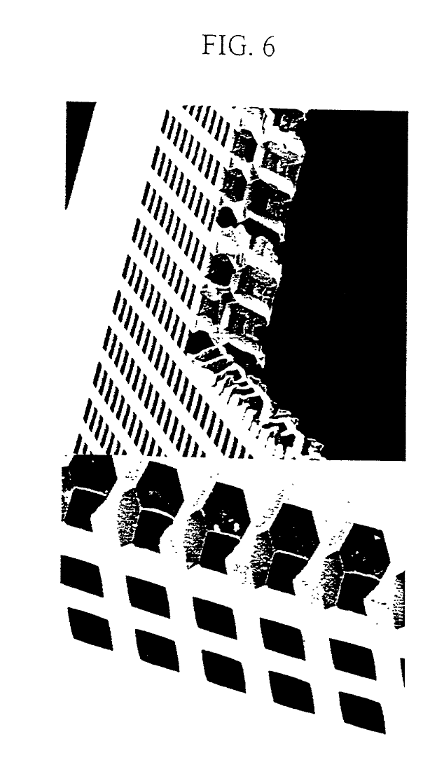

FIG. 3 is a side-angle view illustrating the masking concept of a containment layer 12 with two rigid or soft mask layers 21 and 23. The mask layers 21 and 23 are usually thinner than the containment layer 12. FIG. 3 shows microwells 26 and microfluidic channels 28, within the containment layer. As seen in FIG. 3, the microfluidic channels 28 may have the same or different dimensions and shapes. These cell layers may be applied to only one or bot sides of a containment structure. Equally, the microwells 26 may have different sizes and depths or may form 3-dimensional cavities, as seen in FIG. 6. Both would depend on, for example, the size of the cells or cell clusters, voluminousness of the fluid or extracellular matrix excreted by the cells and transported through the microfluidic channels.

The holes featured as 31 and 33 on the mask layers 21 and 23, respectively, are useful for seeding of different types of cells in the containment layer 12 or any other layer. These holes are connected completely through each layer and correspond to the same features on the containment layer. Holes in layer 23, for example, correspond to the wells on the containment layer 12 but to only a portion of the through holes in the mask layer 21. Therefore, cells seeded on the layer 21 are allowed to pass through the mask layer 23 to the containment layer below to the extent that the cells are seeded only where the two mask layers correlate, as the holes 35, 37 with the well 40 on the containment layer 12. Thus the cells seeded in the hole 35 pass through the hole 37 into the well 40. After one type of cell is seeded from the mask layer 21, mask layer 21 may be removed thereby revealing new hole arrangement in the mask layer 23 corresponding to the well structures in the containment layer 12. Mask layer 23 will remain attached to the containment layer and another type of cell will be applied to the masked containment layer 12 and deposited, for example, through the hole 69 to the well 69 on the layer 12. However, the cells seeded on layer 21 will not be deposited in the well 69 because there is no corresponding hole in the mask layer 21.

After all the cell application and masking steps are complete, the structure seen in FIG. 3, results in a single layer cellular structure of two or more cell types interconnected as shown. Additional masking layers (not shown) can be applied to increase the number of different cells incorporated in a single layer.

FIG. 4 illustrates the process for fabrication of the containment and masking layers as a direct flow chart.

FIG. 4 shows, as an example, a four inch (100) silicon wafer 50 which is first dehydrated in the oven at 150° C. Photoresist is applied to the wafer surface by spin coating the wafer with the resist. The resist-coated wafer 52 is then placed in a contact aligner. Near UV light is used to illuminate a chrome-on-glass mask 51 to expose the wafer with resist 52. The positive resist is solubilized by the UV light where it reaches the polymer surface. The wafers are developed in developing solutions for various lengths of times depending on the resist type and thickness. The wafers now have a layer of patterned polymer over bare silicon 54. The exposed silicon can be dry etched in a plasma to create topological features 53. The silicon wafer with topological features is depicted as a containment or a mask patterned layer 56.

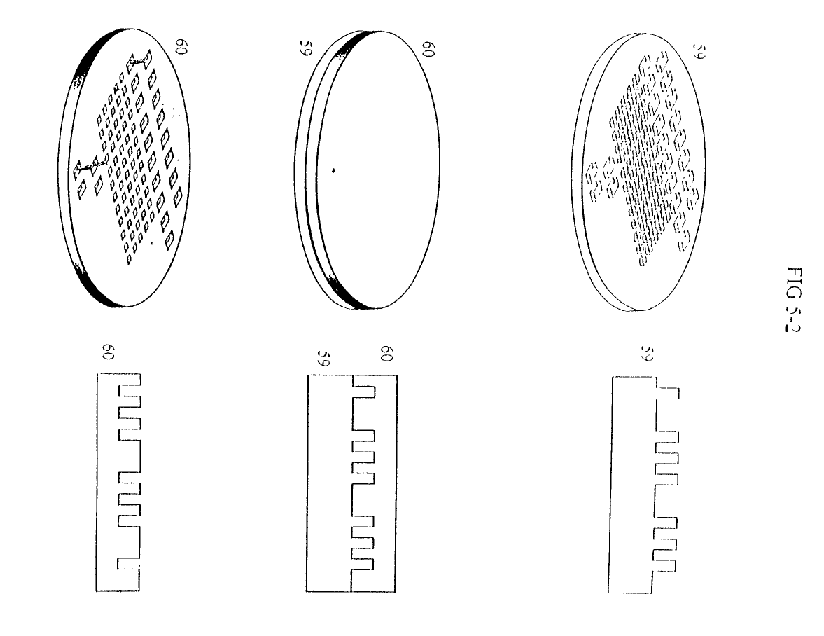

FIG. 5 is a flow-chart illustrating indirect fabrication of a containment layer in biodegradable materials from a silicon based containment layer. The previously fabricated silicon containment wafer 56 is used as a template for a castable polymer 57. The pre-polymer 57 is poured, spun, injected or otherwise spread uniformly on the surface or into the features of the containment wafer 56. The polymer layer 57 is now polymerized in place on the containment wafer 56 and forms a polymerized layer 59 with features in negative to the silicon wafer's features. The polymerized layer 59 can now be cast with a biodegradable material 60. This material is poured, spun, injected or otherwise spread uniformly on the surface of the polymerized layer 59 and then it is gelled, polymerized, or otherwise further processed. Such completed biodegradable containment layer 60 can be removed and now, after processing, has the features of the original silicon containment layer 56.

FIG. 6 is a partial cross-section view showing additional new features of the device, namely three-dimensional cavities of approximately 100 μm of the empty (no cell seeding shown) layers within the device 10. The combination of dry and wet etching in silicon allows fabrication of the three-dimensionally shaped masks and containment structures. The containment structures, in this context, means the structural arrangement of several layers permitting formation of cavities. Three dimensional cavity or container structure as seen in FIG. 6, may increase the yield during cell loading compared to the cylindrical or square cavities described above. The cavities in the containment structures may further interconnect single cells or a cluster of cells by providing horizontal openings between cavities. Such opening may lead to the formation of gap junctions between the cells. Openings or interconnections may further enable the exchange of nutrients, metabolic stimulants, or paracrine control.

In addition to horizontal interconnections between cells, vertical interconnection from one side of the containment layer to the other side or from one containment layer to the adjacent layer may be fabricated by employing combinations of wet and dry etching. Such vertical connections may serve as microchannels through the containment layer wafer. When stacking the containment layers, a connected multi-layer cell network system can be built.

Additionally, the described cell cavity structures enable the use of electrophoretic forces or dielectrophoretic forces or electrokinetic pumping as an aid during the initial cell loading process. Fluids containing cells when pumped with the help of electrokinetic pump may help filling cavities which have at least two openings, one fo the liquid inflow, the other for the outflow. Cells can be captured in the cavity between openings. Opening diameters are designed such that cells can flow into the cavity but not outward.

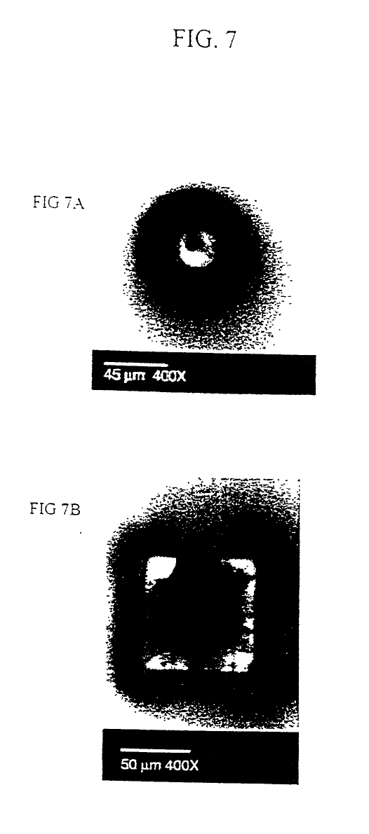

FIG. 7 shows stained chondrocytes deposited in PDMS circular or square wells. FIG. 7A shows placement of one individual cell deposited in a 50 μM-sized circular well 61. FIG. 7B shows emplacement of the cell in the square well 62. The cell is stained with toluidine blue.

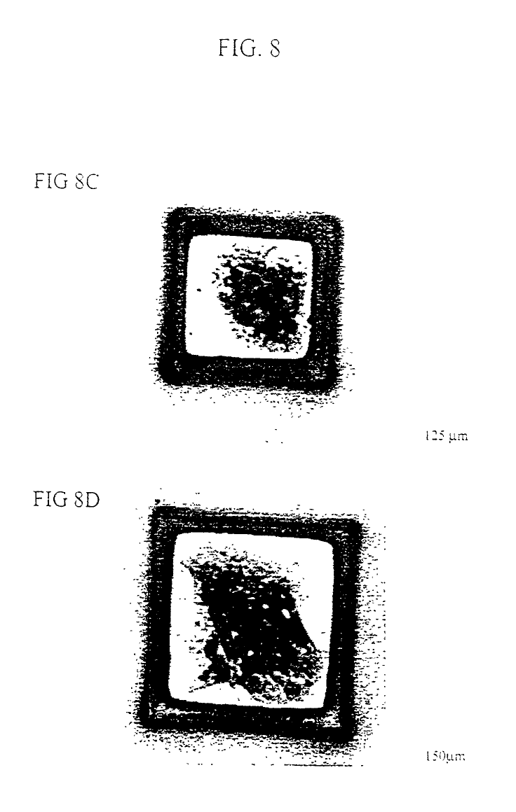

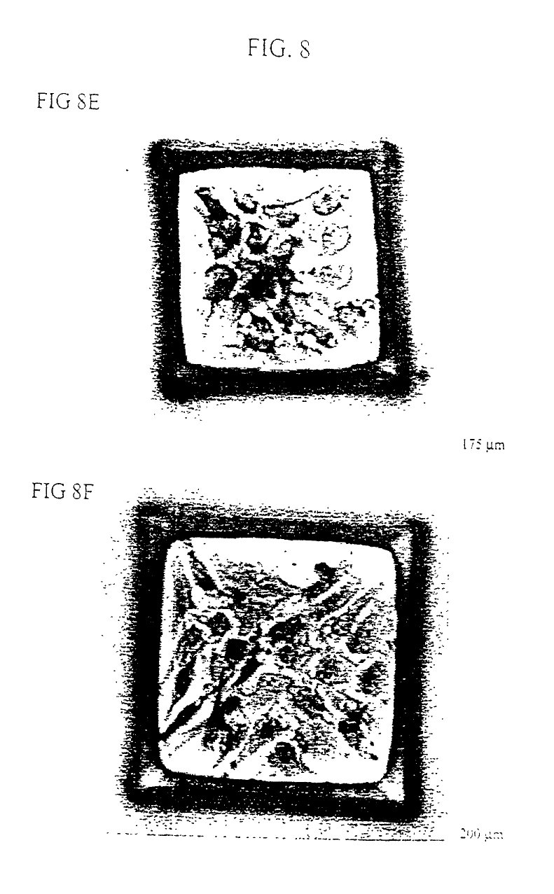

FIGS. 8A-F depict localized chondrocyte deposition within arrays of defined three-dimensional wells. Detailed description of conditions is found in Example 12.

The culture of articular chondrocytes within three-dimensional wells induced the deposition of extracellular matrix around the cells. The extent of the matrix deposition was determined in part by the spacing of the surfaces within the wells that surrounded the cells as well as by the types of attachments made by the cells. The cell attachments to the walls of the wells were dependent on the size of the wells.

In the case of wells designed to just contain a single cell (25 microns), the cells present within these wells were evident using Diff-Quik staining method but little or no extracellular matrix glycosaminoglycan was detected around the cell. In contrast as the dimensions of the wells were expanded to 75 and 100 micrometers, the appearance of chondrocyte-derived extracellular matrix became slightly more prominent as determined by the Diff-Quik method (data not shown) and selective staining of extracellular glycosaminoglycan-rich matrix by Safranin-0 (FIG. 8A, 8B).

In contrast to the smaller sized wells, localization of cells within wells of sizes 125 and 150 micrometers resulted in the appearance of abundant extracellular matrix that could be visualized as surrounding the multiple cells from both the Diff-Quik (data not shown) and the Safranin 0 staining (FIGS. 8C and 8D). In addition, the 125 and 150 micrometer wells resulted in cells that maintained a more rounded morphology with numerous sites where plasma membrane extensions were adhered to the sides of the well.

As the size of the wells increased to 175 and 200 micrometers, the numbers of cells within the wells increased but the deposition of the extracellular matrix by the cells was not apparent either by Diff-Quik (data not shown) or Safranin 0 staining (FIGS. 8E and 8F).

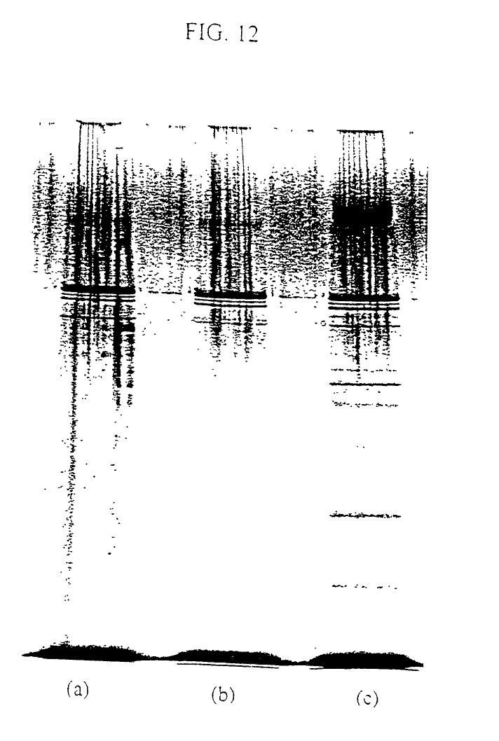

Articular chondrocyte adherence to the planar surface, not in wells, did not show accumulation of extracellular matrix deposition as determined by Safranin 0 staining (FIG. 12).

The data show that localization of chondrocytes within an array of three-dimensional wells can induce the deposition of extracellular matrix surrounding each of the cells or clusters of cells depending on well size. These data confirm a size dependent relationship in the production of cartilage extracellular matrix that correlates with local physical constraints. In this case, the physical constraints were imposed by the geometry of the well, with maximum extracellular matrix deposition occurring at a size range of 125 to 150 microns.

The onset of matrix deposition may then serve as a seeding process by which an accumulation of cartilage matrix can be achieved over a topologic ally designed surface that matches diarthrodial joint geometry. One factor that may influence the extent of matrix synthesis and deposition may involve formation of the cartilage cell plasma membrane contacts within the three-dimensional well. The ability of the cell membrane to bind to the surface while stimulation, influences matrix deposition.

The viability of the cells was preserved in all sizes of the wells suggesting that contact mediated events may be important for intracellular signaling processes that ensure synthesis of matrix macromolecules. The assumption is that addition of mechanical stimuli such as by hydrostatic pressure will significantly increase the production of cartilage matrix macromolecules.

The data presented here show for the first time that articular chondrocytes respond to physical restraints imposed by localized culture within isolated wells on planar surfaces by producing a surrounding extracellular glycosaminoglycanrich matrix.

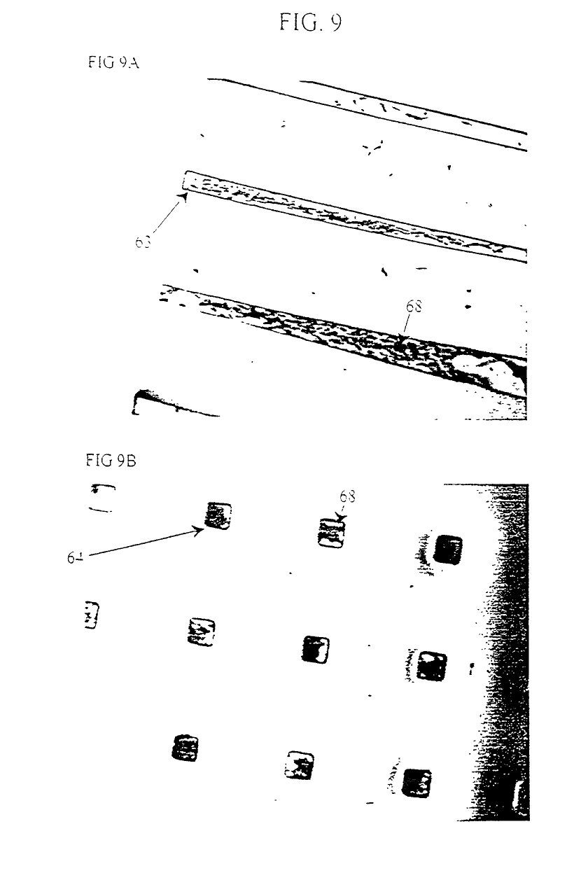

FIG. 9 is an optical image of a PDMS layer on a planar containment layer which creates a clearly visible well structures. FIG. 9A shows longitudinal wells 63 and FIG. 9B shows square wells 64. Individual cells 68 are deposited in the wells 63 and 64.

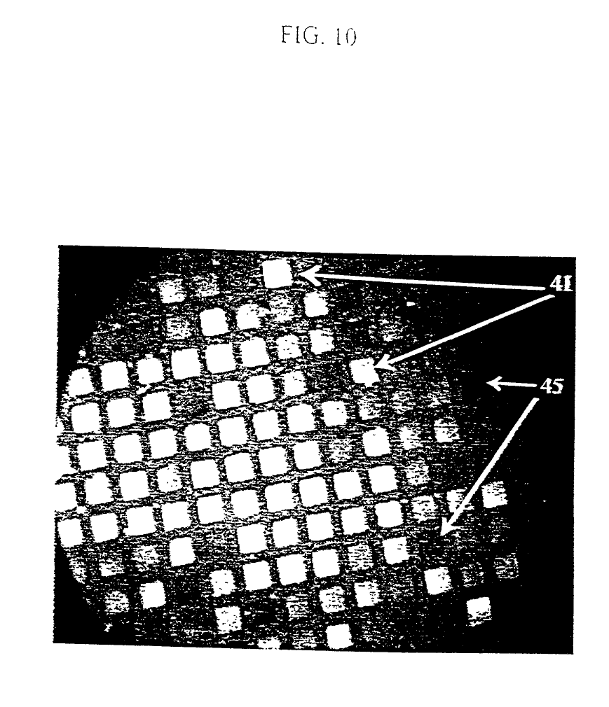

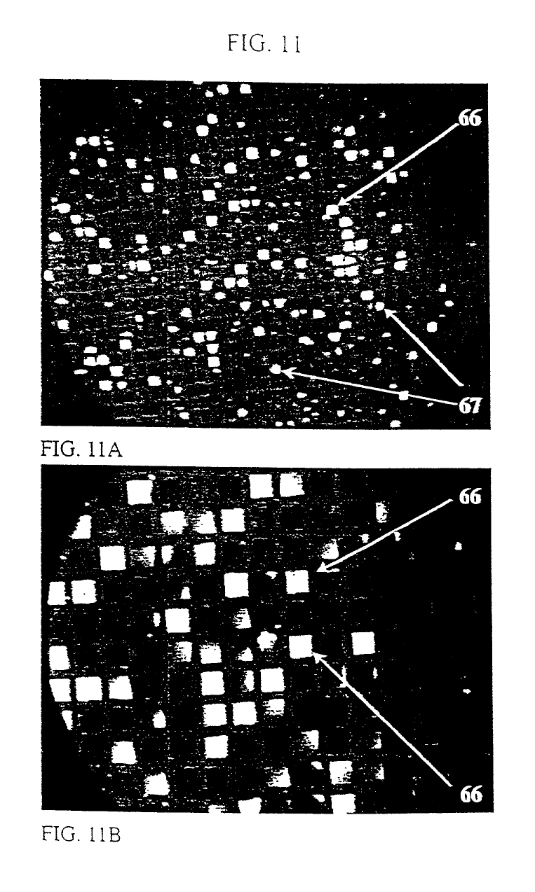

FIGS. 10 and 11A and B show cellular distribution within the construct substrate.

Quantification of cellular adherence, distribution and long term metabolic state of the cells localized within the bioreactor constructs was achieved using vital dye staining and fluorescent microscopy. The culture cells, chondrocytes, fibroblasts and hepatocytes were distributed individually within the substrate constructs containing three-dimensional microwell geometry using the selective masking technology as described.

Following different time periods of culture, namely 3, 10 and 21 days, the distribution, viability and metabolic state of the cells were determined using a commercially available kit from Molecular Probes. The kit utilizes two probes, calcein AM and ethidium homodimer-1. The procedure relies on cellular reactivity to two different types of compounds. One compound, calcein AM, is a fluorescent esterase substrate that when cleaved by intracellular enzymatic activity releases a fluorescent product that is green, seen in the FIGS. 10 and 11 as light gray to white.

As seen in FIG. 10, the filled three-dimensional containment structure with stained fibroblasts clearly shows a cellular distribution of living cells 41 within the wells. The dead cells are seen as dark grey cells 43 and the black wells 45 indicate the empty, unseeded wells.

FIG. 11 shows a low (FIG. 11A) and high power image resolution (FIG. 11B) staining of three-dimensional substrate filled with cells. The brighter spots 66 which are seen in the actual stained figure as green fluorescence indicate viable metabolically active cells. These cells are stained with calcein AM. The second compound, ethidium homodimer-11, only enters the cells through compromised membranes of dead cells and once inside binds with high affinity to the nucleic acid, showing as red staining. In FIGS. 11A and 11B these cells are seen as dark gray 67. The ratio of green to red fluorescence indicates the proportion of cells that are alive versus cells that are dead. The visualization of the fluorescence was achieved using a Zeiss fluorescent microscope with video capture hardware.

The low power image (FIG. 11A) demonstrates that cells are distributed throughout the whole substrate. The variability of signal intensity represents the number of cells within a single microwell with strongest light associated with increased numbers. There were virtually no non-viable cells, with only two isolated spots showing red fluorescence. The high power (FIG. 11B) image shows the range in relative intensities of green fluorescence as visualized by brightness levels. The increased brightness represents live cells distributed in different locations within each microwell and with associated active cell metabolism. No dead cells are seen in the higher power image figure.

FIG. 12 shows results of the analysis of protein expression of hepatocytes alone (lane a), hepatocytes and fibroblasts seeded in three-dimensional device of the invention (lane b) and fibroblasts alone (lane c).

The analysis of protein expression was carried out using polyacrylamide gel electrophoresis under denaturing conditions (SDS-PAGE) for the separation of the individual proteins synthesized by the cells within the three-dimensional bioreactor. The lanes depicted in the gel profile represent the display of protein produced over sixty hours of culture in a serum-free medium. The profile represents fibroblast culture alone in the lane (a) and the combination of fibroblast and hepatocytes in the lane (b) and the hepatocytes alone in lane (c). The protein are detected using silver staining to identify individual species. The size distribution of the protein ranging from greater than 200 kD to as small as 8 kD, as determined by molecular weight markers electrophoresed at the same time as the samples.

To facilitate cell adhesion to the substrate, polylysine was added to the constructs. The prominent band in the upper region of the gel present for all cell samples likely represents slow release of this agent, in spite of washing prior to addition of the cells.

The results of the gel show that the combination of the fibroblasts together with the hepatocytes produces a synergistic increase in the production of proteins as determined by this analytical technique. The size class of proteins produced in the bioreactor construct include a prominent band visual above the putative polylysine band and a number of middle to small proteins detected in the medium below the polylysine band. In some instances the protein represented newly detected forms and in other cases, there was an amplification of proteins produced by the cells cultured alone. These data show that the organization and close apposition of these two distinct cell types within the three-dimensional construct provide a mechanism for modulation of differentiated cell metabolism and function. A repeated gel analysis using a medium sample collected after 80 hours of culture showed identical results.

II. Method for Real-Time Evaluation of Cellular Events

The method utilizing the three-dimensional device of the invention permits determination and evaluation of real-time cellular events, such as gene protein expression, interaction and dependence of individual cells within each organ responsible for secretion and production of hormones and regulatory proteins and intercellular products feedback on the function of cells.

The device permits the distribution and production of cellular products, such as proteins acting as physiological mediators, by a single cell within a three-dimensional array.

The device enables loading of different cell types into different elements of the three-dimensional arrangement. Individual array elements are connected by micro-channels to facilitate exchange of biochemical substances, reagents and electrically charged ions either produced or supplied.

The device permits the localized distribution of cells within a polarized environment to achieve or mimic normal tissue function. For example, the pancreas functions as two organs, one providing an exocrine role and one providing an endocrine role.

The function of the pancreatic cells involves processing of food to nutrients for absorption but also provides the regulatory mechanisms that modulate every other aspect of cellular nutrition. The duality of function of the pancreas is attributable in part to the specialized distribution of the cells within the extracelluar matrix.

The exocrine role of the pancreas provides digestive enzymes. The endocrine role encompasses the synthesis and release of several hormones, namely insulin, glucagon, somatostatin and pancreatic polypeptide.

The method of the invention is illustrated by using the device of the invention as a simulator for replacement of pancreas. The endocrine role of pancreatic cells is supported by the device of the invention by providing a means of establishing the device in lieu of pancreatic islets. Pancreatic islets are micro-organs composed of four cell types, called the A, B, D and F cells. The endocrine function of the islets depends on an ordered distribution of the cells with an organizational polarity of inside to outside. The defined distribution of cells, in turn, leads to function through the directionality by which the product of the islets are modified prior to release into the organism.

In this respect, the F cells exist in a posterior lobe of the pancreas and secrete the pancreatic polypeptide. The B cells are located in the tail and anterior portion of the pancreas and secrete insulin. The A cells constitute about 20% of the cells and produce glucagon while the D cells produce somatostatin. The function of the pancreas depends on close proximity of the different cell types to each other as their products effect each other.

The increase in blood glucose following a meal stimulates B and D cells to produce insulin and somatostatin. The first target cell of insulin are the pancreatic A cells which reduce secretion of glucagon. The somatostatin released by the D cells also act to reduce glucagon release by the adjacent A cells. However, if amino acids are high in the blood the levels of glucagon will remain elevated.

The device of the invention provides a substitute for pancreas in that it permits positioning of the four types of cells, namely, cells A, B, D and F in close interrelations in which they exist in the pancreas.

The proximity of the cells within the device matrix dictates the cellular responsiveness through these paracrine interactions. The device provides the microfluidic channeling and the surface adhesion molecules to recapitulate the islet cellular geometry. The device comprising all four types of pancreatic cells can be surgically implanted in locations where the release of pancreatic cell products will enter the normal circulation through vascular and lymphatic uptake.

Similarly to the pancreas, the function of other organs may be successfully simulated by designing the three-dimensional device which would interdependently connect different types of cell in close-circuit like fashion where the cellular products secreted or produced by the cell would perform the same function as the same cells perform in the intact organ.

III. Tissue and Organ Function

Tissue function results from the interaction between differentiated cell types that express specialized cellular products which are regulatory to other cells. Production of organ or tissue specific products involves the coordination of diverse regulatory signaling pathways acting through direct and indirect intercellular communication. In case of exocrine and endocrine glandular tissues, for example, the cells are typically divided into two classes, epithelial and stromal cells. Each set of cells is instrumental in providing the physiological cues that result in steady state metabolic activity of both cell types.

The communication processes between the cells involve direct cell contact at the level of adjacent plasma membranes where pores exist for transfer of materials between the cells, such as with gap junctions, synapses, etc. In other cases, the cells form tight junctions between adjacent membranes, producing barriers so that uptake and release of metabolites and cell metabolism products are directed to either the apical or basal surface of the cells.

Different cell types usually release different soluble mediators that then modulate the metabolism of neighboring cells. The soluble mediators act either as autocrine factors that modulate the metabolism of the same cell producing the mediator, paracrine factors that interact with neighboring cells, or as endocrine factors that are spread throughout the organism by the blood and lymphatic systems.

In highly specialized organs such as the liver, pituitary and pancreas, the tissue level of organization extends into areas of zonal specialization where multiple cellular interactions generate and control organ functions. The pituitary gland, for example, secretes a large number of hormones that activate many peripheral endocrine cells such as those of the adrenal glands, thyroid, testes and ovaries.

To illustrate the invention, several examples of tissue malfunction that require intercellular communication for restoration of function are presented. The pituitary, pancreas, liver and brain are exemplary organ systems in which production of cellular products by multiple layered arrays of cells deposited within the three-dimensional device of the invention provides restoration of function. Chondrocytes are exemplary cells which produce proteoglycan and collagen when loaded onto the device of the invention.

A. Pituitary Gland

Anatomically the pituitary is divided into two distinct functional parts. The anterior pituitary (adenohypophysis) is an epithelial-derived tissue that has three distinct components each with specialized cell types. The posterior pituitary is composed of neuronal tissue with accompanying glial cells that are subdivided into three lobular components.

The anterior pituitary is composed of five distinct types of endocrine cells that are unevenly distributed within the tissue. The somatrophs secrete growth hormone, betalipotrophin, and other hormones. The thyrotrophs release thyroid stimulation hormone. The gonadotrophs secret gonadrotropic hormones.

B. Pancreas

The pancreas provides two major functions for the organism as already briefly described above. The alkaline ? secretions that are released following a meal aid in the breakdown of proteins, carbohydrates, fats and nucleic acids. In addition, the gland produces two hormones, insulin and glucagon, that regulate carbohydrate metabolism.

The pancreas is composed of a loose connective tissue stroma that is interposed among the epithelial acinar cells. The acinar cells have polarity so that secretions are released into a series of intralobular ducts that merge together prior to emptying into the lumen of the duodenum.

The endocrine function of the pancreas is carried out by clusters of cells called the islets of Langerhans nonuniformly distributed within the gland. The islets are composed of a collection of four different cell types called cells A, B, D and F. These cells are distinguished by size, electron density and granule content. The individual cell types are distributed within the tissue in a non-uniform pattern.

The islets release insulin, glucagon and somatostatin. The cells of the islets are clustered together and the groups are scattered among the acinar cells.

The A-cells are associated with release of glucagon, the B-cells release insulin, the D-cells release somatostatin and the F-cell release the pancreatic polypeptide. The close proximity of each of the cell types results in an interdependence for the regulation of the products of the Acells, the B-cells and the D-cells.

The F cells which secrete pancreatic polypeptide and are primarily found in islets within the posterior portion of the gland account for 80-85% of the islet cells. Islets in posterior portion of the gland also contain insulin secreting B cells (15-20%) and a small proportion of A cells (0.5%). The posterior portion of the gland is separated from the anterior part of the gland by fibrous partition. The anterior portion of the pancreas consists of islets that are composed of 70-80% of insulin secreting B cells, 20% A cells that secrete glucagon and 3-5% of D cells that produce somatostatin. The spatial distribution of the cell types supports the release and transport of insulin from the middle zone of the islets to the peripheral border of the islets where insulin modulates glucagon release from the A cells.

The physiological function of the islet in regulating glucose levels in the presence and absence of immediate food intake requires products from all four cell types within the islets. The coordinated regulation of the levels of glucagon, insulin and somatostatin then contributes to physiological homeostasis with respect to glucose metabolism in the whole organism. Loss of this functional regulation is the basis of diabetes.

The device of the invention reproduces the geographic and geometric distribution of the cells to maximize normal physiological function through a combination of five layered arrays. The outermost two layers of the pancreas-mimicking device are composed of cells isolated from the posterior regions of the pancreas, thus providing the F cell component. The middle two layers consist of cells isolated from the peripheral ring of islets localized within the anterior portion of the pancreas, these cells provide the A and D cell function. The inner most layer consists of cells isolated from the middle zone of the islets from the anterior portion of the pancreas, providing the B cells function.

The construction of the devices according to the invention permits the patterned distribution of the pancreatic cells within the wells of the several containment layers that are interconnected by directional microfluidic channels. The availability of each of the four cell types within close approximation of each other facilitates the normal physiological feedback regulation that is necessary for fine control of glucose concentration in the organism.

The microfluidic channels provide the polarity of flow so that a blood vessel can be anastomosed to the support layer and through the support layer into the device interior. Flow of the blood or any physiologically acceptable fluid occur in a radial fashion permitting distribution of insulin from the B cell to the A and D cells, where glucagon and somatostatin release is modulated and finally to the F cells where the pancreatic polypeptide is released.

Outlet port anastomosed to the venous blood circulation removes product of cellular metabolism of cells A, B, D and F into the circulation where they perform their normal function during digestive process.

In alternative, the device as described above is stand alone and produces the insulin, glucagon, somatostatin and pancreatic polypeptide which is harvested and used as appropriate.

Both arrangements provide either a three dimensional cell-based physiological sensor for the restoration of damaged or destroyed pancreatic islet of Langerhans for the treatment of diabetes or a protein factory for production of pancreatic products.

C. Liver

The liver is the largest compound gland in the body and is the primary site for detoxification of circulating substances while serving as a reservoir of carbohydrates, a source of glucose, plasma proteins and lipoproteins and as a secretory gland for release of bilirubin, IgA and bile salts.

Physiological function of the liver is accomplished by hepatocytes. Hepatocytes, a major cell type of the liver, are localized within the tissue as zones of specialization.

The hepatocytes are secretory epithelial cells organized into rows that converge toward a central vein. The hepatocytes border on sunusoids where the cell surface is exposed to blood plasma. The proximity of the cells to blood plasma facilitates the uptake of compounds and release of secretory products.

The hepatocytes are organized into functional units known as acini. Each hepatic acinus is composed of a hepatocytes that are present as plates of approximately 20 cells that are one cell layer thick so that access to both venous and arterial blood is accomplished. The organization of the cells creates a solute concentration gradient so that blood-borne solutes move unidirectionally through the cells in a sequential order. The first hepatocytes remove solutes that are low molecular weight metabolites such as ammonia, oxygen and bile acids whereas the secondary and tertiary level hepatocytes receive solutes bound to albumin. The gradient of metabolic products created through the cellular organization appears critical to liver function.

Hepatocyte function is increased by a co-culture system in which the hepatocytes are placed in proximity to fibroblasts. The hepatocyte-fibroblast cell-cell interaction results in increased albumin production and urea synthesis when compared to the cultures of hepatocytes maintained alone.

The device of the invention comprises a three-dimensional spatial arrays of hepatocytes organized in the same manner as are hepatocytes within the liver. Due to this arrangement, a reconstruction of liver function is achieved according to the cellular zonal specialization.

The device of the invention consists of wells in nanometer sizes that permit deposition of cells in a formation of groups of twenty cells. Such group of cells permits establishment of intercellular tight junctions between individual cells. The groups of cells are interconnected by a radial pattern of canals to achieve free distribution of metabolites among the cells. In the liver simulated device the hepatocytes are deposited on three inner plates located within a layered array of five plates. The outer plates contain stromal fibroblasts to provide paracrine factors, such as hepatocyte growth factor and IL-6 that enhance hepatocyte viability and production of liver protein, such as for example production of albumin.

D. Brain

A number of disorders of the human nervous system can be linked to the selective destruction of nerve cells so that interconnected signaling is disrupted. These diseases include abnormalities of movement and posture such as those that occur in Parkinson's disease, Wilson's disease, Huntington's chorea and manifestations of Jakob-Creutzfeld disease. In Parkinson's disease, the destruction of the neurons of the substantia nigra and the loss of the connection with nigrostraital pathway result in decreased dopamine production. The production of dopamine is a critical component in the regulation of muscle contraction where the molecule serves as a neurotransmitter substance.

Efforts to treat the Parkinson's disease are complicated by the loss of intracellular monitoring that maintains the balance between normal movement and slowness or arrhythmic movement due to overshooting of the active drug.

Application of the three-dimensional nanowell array is useful for the restoration of function in Parkinson's Disease through the combination of two cell types. The device consist of three cell types, one producing and releasing L-dopa, the substrate for the neuron production of dopamine. The second cell types are fetally derived neurons that are permitted to interconnect with a third set of cells, basal ganglia cells. The purpose of the device is to restore a system of cell interactions through neuronal mimicry that culminate in the regulated release of levels of the basal gangia cell derived neurotransmitters, acetylcholine, 5-hydroxytryptamine, dopamine, γ-aminobutyric acid and glutamic acid.

E. Articular Cartilage

A device consisting of neurons capable of producing dopamine distributed within microarrays connected with channels to regulatory neurons that process the dopamine into active neurotransmitter substances is effective in mitigating the symptoms of these neuropathies.

Articular cartilage functions as the primary mechanical interface for distribution of relatively large loads (4 to 7 times body weight) across the major diarthrodial joints. Articular cartilage is a tissue that is a composite material that includes numerous cells surrounded by a relatively abundant extracellular matrix.

The functional capacity of articular cartilage is derived from the physical properties of the two major macromolecules that comprise the bulk of the extracellular matrix, the type II collagen and the large aggregating proteoglycans, the aggrecans. The type II collagen is organized within cartilage in fibrillar bundles that are composed of individual fibrils of collagenous peptide cross-linked to each other by covalent bonds. The type II collagen network provides cartilage with tensile properties. The aggrecans are macromolecules that exist as protein core to which two types of glycosaminoglycan chains are co-valently attached at serine and threonine residues.

The glycosaminoglycan chains of the aggrecans are repeating sequences of disaccharide, of which one sugar residue (in the case of chondroitin 4, or 6) or potentially both (in the case of keratan sulfate) are sulfated. The strong electronegativity and the hydrophilic character of the aggrecans due in part to sulfation contribute to a high water content and strong resistance to flow. The aggrecan ensure a tissue low permeability and a residual swelling pressure that provides the tissue with compressive resilience.

One major challenge in the process by which cartilage repair and regeneration can be achieved is the reestablishment of a contoured surface of extracellular matrix that would serve as a replacement of diseased and destroyed tissue. An approach for deposition of single or small groups of cells within a defined pattern to facilitate deposition of extracellular matrix would fulfill a major need for production of a cartilage matrix. Currently, culture of chondrocytes as planar high-density monolayer cultures results in release of the extracellular matrix macromolecules into the surrounding culture medium and little cell-associated extracellular matrix.

This study utilizing a device of the invention tested whether chondrocyte matrix deposition might be influenced by deposition of the cells within formed wells having a predetermined depth, length and width on a planar support structure. The experiment tested the effects of selective adherence of isolated articular chondrocytes within wells of increasing areas that permitted an increase in cell number from 1 to approximately 50 on induction of extracellular matrix formation.

Results are described in Example 11.

### Utility

The current invention has broad medical and scientific usefulness. In particular, the three-dimensional device of the invention are useful for therapeutic purposes, such as, for example, permanent or temporary replacement of the diseased tissue or organ or replacement of dysfunctional or malfunctional tissue or organ.

The design and materials used for fabrication of the device provide actual medical implant that consist of entire tissues, organs or organ systems.

The device may further be used as a research structure for co-culture or for multiple culture of several types of cells for studying cell systems.

The device is also useful for diagnostic purposes whereby the functionality of each type of the cells is tested within the system.

Additionally, the device may be used for testing activity and efficacy of pharmaceutical agents.

### EXAMPLE 1

### Lithographic Process for Fabrication of Containment and Masking Layers

This example describes the lithographic process for production of containment and masking layers.

A modified lithographic procedure consisted of baking the silicon wafers for 150° C. for 30 minutes. The wafer was placed in the Silicon Valley Group (SVG) Photoresist Spin Coater. The wafers were exposed to HMDS for 5 seconds followed by a 5 second bake at 125° C. This cycle was repeated 3 times. SPR220-7 was dispensed onto wafers spinning at 500 rpm. The wafers were then spun at 3500 rpm for 30 seconds resulting in a 7 μm uniform film of SPR220-7 resist on the wafers. The wafers were then baked on a hot plate for 300 seconds at 90° C. The wafers were exposed to UV light in a Silicon Valley Group 620 Contact Aligner through a photomask of chrome on glass for a length of 7.7 seconds at 16 mw/cm², and then developed on a Silicon Valley Group Resist Developer that develops using Shipley LDD-26W for 3 cycles of 100 seconds followed by a 20 second rinse. The wafers were dry etched to impart topological features according to masking design. Dry etching consists of exposing the wafers to a cycle of SF₆ plasma followed by a C₄F₈ passivation layer. This was performed in the Surface Technology Systems (STS) Multiplex ICP Deep Reactive Ion Etcher to etch at anywhere from 0.5 μm/minute to 5 μm/minute depending on the geometry of the etched structure and the etch program chosen. The photoresist was removed in a piranha solution of 9:1 sulfuric acid: hydrogen peroxide. The wafers were patterned in silicon with topological features.

### EXAMPLE 2

### Polydimethylsiloxane Based Device

This example describes fabrication of a polydimethylsiloxane (PDMS) variant of the three-dimensional device.

Templates for cell isolation arrays are constructed using standard photolithographic methods as described in Example 1.

Silicon wafers are cleaned of residual organic material in a piranha solution (9:1, H₂SO₄:H₂O₂) and then rinsed for six cycles in distilled water. The wafers are dehydrated for 1 hour at 150° C. and then developed and rinsed producing the desired template.

A 10:1 ratio of heat curable silicone elastomer (polydimethylsiloxane, PDMS) Sylgard 182 (Dow Corning) and curing agent are mixed thoroughly and evacuated to ensure complete mixing and remove air bubbles. This solution is vacuum cast between the photoresist-on-wafer and a gasketed glass wafer to produce PDMS layers on the order of 20-40 μm thick containing through features in negative to wafer templates. These wafer PDMS sandwiches are cured at 90° C. for 1 hour. The PDMS layers are removed from the wafers with the help of the gasketing layer. They are then placed in an oxygen plasma cleaner (Harrick Scientific) for approximately 40 seconds.

Oxidation introduces silanol (SiOH) groups at the surface of the naturally hydrophobic polymer and makes the surface of oxidized PDMS resemble the surface of silica in its wettability properties. This step creates a highly reactive surface that can then be surface grafted with cytophobic polymers to introduce chemically defined regions of cell repulsiveness, surface modified to make less adhesive to other PDMS surfaces, or can be attached irreversibly to glass or other hydrophilized PDMS layers to build up layers of containment structures.

### EXAMPLE 3

### Surface Grafting of Cytophobic Polymer

This example illustrates surface grafting of cytopholic polymer.

The oxygen plasma or air plasma oxidation of the PDMS or glass introduces surface silanol groups that are very reactive.

The process of grafting cytophobic polymers such as (PEO) polyethylene oxide or polyethyleneglycol (PEG) consists of reacting an intermediary polymer polyethyleneimine with the hydroxyl groups on the surface of the glass or PDMS followed by reacting the exposed amine groups with an epoxide linked to a polyethylene oxide or glycol.

In detail, the PDMS or glass surface is oxidized for three to five minutes in a Harrick Scientific Plasma Cleaner at a high RF power setting. The polymer or glass item is then placed in a solution of 3% PEI (BASF) in 0.05 M sodium carbonate buffer (˜10.3 pH) for 3 hours and kept at 45° C. The item is then washed in deionized water and immersed in 10% weight/volume PEG or PEO (PEG epoxides of various molecular weights from Shearwater Polymers) in 0.05 M carbonate buffer and kept at 40° C. for three hours.

### EXAMPLE 4

Surface Grafting of Cytophobic Silanes

This example describes surface grafting of cytopholic silanes.

The surface is exposed to plasma in the same manner as in Example 4 for similar amounts of time. Then the item is placed in a desiccator with 200 μL of noctadecyltrichlorosilane (n-OTS) or 13F in paraffin oil. The desiccator is evacuated and back filled with N₂ nitrogen. The evacuation and backfilling with nitrogen is repeated and then the items are kept under vacuum for 2 hours. The silanes in the vapor phase will self assemble on the surface of the oxidized glass or polymer. After 2 hours the silane surfaces are cured in an oven at 120° C. for 5-20 minutes to complete the self assembly process.

### EXAMPLE 5

### Glass-Based Device

This example describes fabrication of a glass variant of the three-dimensional device of the invention.

Silicon masking wafers are cleaned using a typical prediffusion cleaning process (9:1 H₂SO₄H₂O₂ 10 minutes, 5:1:1 H₂O:H₂O₂:HCl 10 minutes, 50:1 Buffered Oxide Etch 30 seconds). Then approximately 5 μm of silicon nitride are grown uniformly on both sides of the wafer.

The wafers are then dehydrated for approximately 30 minutes at 150° C. The wafers are spin coated with 7 μm of positive resist (Shipley, SPR220-7). The wafers are placed in a mask aligner (Karl Suss MA-6 Contact Mask Aligner) and the backside is lithographically patterned with large regions. These large regions are dry etched (Drytek 100 series plasma etchers) to remove the nitride masking layer and then the exposed silicon is wet etched using KOH creating thin membrane regions to maximize the accessibility to the containment structures by the cells.

The front side of the wafer is now photolithographically defined and dry etched (STS Deep RIE Etcher) to produce through holes in the membrane regions.

100 mm rounds of borofloat glass with a layer of 1500 Å of amorphous silicon are obtained. These Am—Si on glass wafers are lithographically patterned to define regions of cell containment structures and vias for cell-cell communication and passage of cell products. The Am—Si layer is dry etched away and then the exposed glass regions are subsequently wet etched to depths appropriate for the tissue of cell being cultured.

Am—Si layer is etched for 2 minutes exposing the glass beneath in a Drytek 100 Series Plasma Etcher with a chamber pressure of 100 mT and gas flows of 100 Standard Cubic Centimeters per Minute (sccm) SF6 and 70 sccm C2C1F5 (Freon 115) at an RF power of 55 W. Some regions of the wafer can then be protected from the impending wet etching process by isolating non-etch areas with dicing tape. The exposed bare glass regions are subsequently wet etched in 49% Hydrofluoric Acid 51% water. The approximate etch rate for this process is 7 μm/minute. Etch times are adjusted to achieve the desirable depth in the glass.

For single cell studies, typical depths of microwells are on the order of the cell size, approximately 20 to 40 μm. For tissue culture and via construction, the topology of the substrate wafer can be engineered for optimal effectiveness.

The masking silicon wafers are aligned to the substrate glass wafers in a mask aligner. The wafers are mechanically and reversibly fixed to one another by mechanical clamp. The wafers are mechanically and reversibly fixed to one another through the use of an alignment chuck that can facilitate alignment while keeping the wafers in conformal contact with one another for cell plating purposes. The wafers can also be mechanically clamped together with clips designed for this purpose that will interface with the wafer alignment chuck from the Electronic Visions 620 Contact Aligner. The wafers can simply reversibly adhere to one another using tapes or other adhesives. Cell adhesive proteins such as polylysine (0.1 mg/ml in phosphate buffered saline) Fibronectin at 10 μg/ml in phosphate buffered saline. The glass substrate layer is immersed in a solution of cell adhesive proteins such as polylysine (0.1 mg/ml) or fibronectin (10 μg/ml). These proteins adsorb at the surface and promote cell attachment.

The substrate-mask stack was then exposed to cells either under gravity or through centrifugation to increase the effectiveness of gathering cells in the containment structures of the substrate wafer. The cells are allowed to adhere 1-4 hours and then one masking layer is removed revealing new regions of the substrate wafer. Another variety of cells can now be deposited into these newly revealed regions. In this way, multiple cell types can be incorporated in patterned fashion in each layer. These layers can be combined to create functioning tissues with microfluidic channels between the layers to allow for the passage of cell products and nutrients.

### EXAMPLE 6

### Isolation of Cells for Depositing

This example describes the process used for isolation of cell from tissues for deposition within the three-dimensional device.

Tissue appropriate to the tissue which the device being constructed is simulating, was obtained and enzymatically digested with collagenase (1 mg/ml) and tissue culture medium combining 25 μg/ml gentonycin at 37° C. for 36 hours to reduce the tissues to single cells. The cell slurry was passed through a syringe filter of sterile nylon mesh with a pore size of 50 μm to remove undigested tissue. The cells were collected by centrifugation at 600× g for 15 minutes and resuspended in phosphate buffer saline (PBS). The cells were then be distinguished by type and prepared for seeding on the masked structures.

The isolated cells were centrifuged onto the surface of the fabricated masked containment structures. The constructs were maintained statically for approximately 1 hour to permit cell attachment to the substratum. Appropriate masking layers were removed and subsequent types of cells centrifuged into the newly revealed section of the containment structures.

### EXAMPLE 7

### Articular Cartilage Cells

This example describes testing of articular cartilage cells for depositing within the device of the invention.

Articular cartilage as an initial cell type was obtained from normal bovine radiocarpal joints. Cartilage was dissected as full-thickness pieces and washed three times in PBS. The pieces were then enzymatically digested at 37° C. in a solution of 0.25% trypsin-EDTA after 1 hour the pieces were washed in PBS. A sterile filtered solution of 1 mg/ml type II collagen and I mg/ml type IV collagenase in DMEM F12 was added and allowed to digest for 2 days in the incubator.

The cell suspension was collected through a syringe filter of sterile nylon mesh with a pore size of 50 μm for 15 minutes. The cells were collected by centrifugation and washed in PBS for three cycles and resuspended in serum free DMEM/F12 medium containing 50 μg/ml gentamicin, lipid and asorbic acid.

The isolated cells were passed through an air spray gun to create nanodroplets with an estimated volume of 50 to 200 nanoliters onto the surface of the fabricated picovolume wells to disperse one cell per well. The microwells were maintained statically for approximately 1 hour to permit cell attachment to the substratum. Additional serum free media was added and the cultures were placed on an orbital shaker in an incubator at 37° C. and 5% CO₂ for 24 hours to promote further dispersion of the cells into the wells. The constructs were then rinsed and DMEM/F12 with 10% FBS was added. The constructs were exposed to mechanical stress fields.

### EXAMPLE 8

### Testing of Mechanical Loads

This example describes testing of articulate cartilage cells using the device of the invention.

To study the effect of intermittent hydrostatic pressure on normal human chondrocytes, a commercially available stainless steel pressure vessel interfaced to a servo-hydraulic loading instrument (MTS) were used for quantitative loading.

The loading protocol provides for complete evacuation of air from the system so the application of pressure is solely hydrostatic. The cells were maintained in flexible, heat sealed containers in which the picovolume well arrays are completely immersed in culture medium. The fluid filled loading vessel was maintained at 37° C. During the loading period, cells could be viewed to assess the level of expression of indicators such as green fluorescence protein (GFP). At the conclusion of the loading period, the cells were harvested for analysis of proteoglycan and collagen synthesis or prepared for immunohistochemical analysis of matrix synthesis using 3% paraformaldehyde in PIPES buffer.

To study the effects of Shear stress, the elastomer substrates were adhered to glass slides. An actuator was used to apply a shear to the exposed top surface of the individual cells. Similarly, levels of expression during the stress application and at the conclusion were analyzed.

### EXAMPLE 9

### Quantification of Extracellular Matrix Production

This example describes quantification of extracellular matrix production.

For immunohistochemical analysis of matrix formation, following the fixation in paraformaldehyde for twenty minutes, the cells were rinsed three times with phosphate buffered saline (PBS).

Each culture sample was treated with monospecific polyclonal antibodies prepared in rabbits against type II collagen and proteoglycan core protein. In all cases, rabbit non-immune serum served as the rabbit IgG control. After specific labeling for 1 hour at 25° C., the rabbit antibody was removed and the cells were then treated with an FITC-labeled goat antibody preparation specific for rabbit IgG. Following a period of thirty minutes for binding, the cells were washed in PBS and overlaid with a solution of PBS/glycerol (1:1). The distribution of extracellular matrix molecules between the outside and inside of the chondrocytes was determined by permeabilizing the cells to antibody using the nonionic detergent, Triton X-100. The analysis of the extracellular macromolecules was carried out using phase contrast and fluorescence microscopy to determine the deposition of antibody to large aggregating proteoglycan and type II collagen in the cellular arrays.

The fluorescence of the individual cells was quantified using digitization techniques for analysis of the light emissions. The results obtained form the immunochemical analysis demonstrated on a per cell basis, what effect application of hydrostatic pressure had on extracellular matrix deposition of proteoglycan and collagen.

### EXAMPLE 10

### Investigation of Intracellular Morphologies

This example describes the process used in investigation of intracellular morphologies.

The localization of individual cells allowed for the determination of intracellular pathways for biosynthesis and the responses at the subcellular level to mechanical stimuli. Through the use of a specialized AFM tip, the electrical potential of cell surfaces was imaged and through this step molecular events following mechanical stimulation and changes in intracellular organization and surface morphology were determined.

### EXAMPLE 11

### Localized Chondrocyte Deposition Within Arrays of Defined Three-Dimensional Wells for Induction and Deposition of an Safranin-0 Positive Glycosaminoglycan-Rich Extracellular Matrix

This example describes studies performed to determine induction of the deposition of extracellular matrix surrounding the cell or cell cluster and its dependency or the well size.

The articular chondrocytes were isolated from human osteoarthritic cartilage samples using collagenase digestion at a concentration of 1.2 mg/ml in culture medium (DMEMIF12, Gibco) and overnight incubation at 37 C in a humidified air and 5% carbon dioxide. Cells freed of extracellular matrix were collected by centrifugation at 600-× g for twenty minutes. The cells were resuspended in forty milliliters in Dulbecco's phosphate buffered saline (calcium and magnesium free) and the cells collected by centrifugation. This step was repeated three times to remove collagenase.

The cells were then suspended in culture medium (DMEMIF12) and the number determined by counting in heniatocytometer in the presence to Trypan blue to determine viability (>90%). The cells were diluted into culture medium (DMEMIF12 containing 10% fetal bovine serum (dialyzed and heat inactivated) for plating on prepared silicon containment layers containing wells of varying dimensions.

The cells were applied to the silicon containment layers for varying times (2, 4, 12 hours) with gentle agitation to deposit the cells within the wells and prevent adherence of cells within the interwell surface spaces.

After an attachment period, the containment layers were then maintained in culture for periods of 3, 7 and 10 days prior to fixation with 10% buffered form align and staining of the extracellular matrix deposition using Safranin 0. Alternatively, the cells and the surrounding extracellular matrix was stained with a commercially available staining kit (Diff-Quick, Sigma Chemical, St. Louis, Mo.). Cell viability within the wells was determined by brief exposure to Trypan blue (0.04% in saline).

### EXAMPLE 12

### Seeding and Manipulation of Different Types of Cells Within the Three-Dimensional Device

This example describes methods used for seeding different types of cells within the wells of the three-dimensional device.

Substrates and masks were aligned manually using a micromanipulation stage. They were mechanically and reversibly attached to one another. The mask substrate combination was autoclaved for sterility. Secondary masks were fabricated from thin layers of polydimethylsiloxane (PDMS). The thin secondary masks were sterilized in 70% ethanol for at least 30 minutes. The sterile PDMS secondary masks were washed thoroughly and then floated onto the primary masks were allowed to adhere reversibly to the primary mask and secondary mask combination was washed thoroughly in phosphate buffered saline and the microwells were mechanically wet by pipetting up and down. The PBS was removed and the construct was rinsed with a serum free media DMEM/F-12 supplemented with gentamicin, selenium, dexamethasone, insulin and proline.

Mouse NIH 3T3 fibroblast and Mouse hepatocytes were obtained from American Type Culture Collection. The cells were treated with trypsin to remove them from their culture plates. The cells were then washed thoroughly and resuspended in the above mentioned serum free medium. The hepatocytes were plated onto constructs after no more than four passages by placing a bead of medium containing cells in the center of the device. Similarly on the other control substrate fibroblasts were plated. These substrates were allowed to adhere for 24 hours after which the effluent bead was drawn off and the surface was rinsed with serum free media. The secondary mask was removed from the test construct with sterile forceps. Fibroblasts were plated onto the surface for the primary mask and filling the newly exposed passages to the substrate wells. The fibroblasts were allowed to adhere for 24 hours and the effluent was removed and the mask was rinsed with PBS.

The masks were removed from the substrates observing sterile technique. The substrates were briefly rinsed in PBS and the placed in new tissue culture containers with fresh serum free media. After 60 hours in culture a sample of the medium was collected and fractionated on a Nupage bis-tris electrophoresis gel to determine protein content.

What is claimed:

1. An implantable three-dimensional device or a standalone bioreactor or device for anatomical, histological and spatial inter-reaction of one or several types of cells, cell clusters or tissues, said device comprising:
a) a support layer; and
b) a multiplicity, from one to several thousands, of containment layers comprising a multiplicity of wells interconnected horizontally within one containment layer, vertically or through inclined microfluidic channels with other containment layers.

2. The device of claim 1 additionally comprising one or more permanent or removable masking layers for cell deposition into the wells of the containment layers, said masking layers comprising openings having a pattern corresponding to several or all wells positioned on the containment layer immediately below the masking layer.

3. The device of claim 2 wherein the support layer is fabricated of glass, silicon, hardened polymer, titanium, film or porous material having a smooth or etched surface.

4. The device of claim 3 wherein said support layer additionally comprises a gated or nongated inlet port and a gated or nongated outlet port and wherein said support layer is flat or have a rising edge or a rim to form a containment structure comprising a containment cavity for emplacement of a multiplicity of containment layers within said cavity.

5. The device of claim 4 comprising the multiplicity of containment layers emplaced within the containment cavity of the support layer.

6. The device of claim 5 wherein the containment layer is fabricated from a biocompatible biodegradable material, a biodegradable hydrogel, or a rigid nonbiodegradable material.

7. The device of claim 6 wherein said rigid nonbiodegradable material is a glass, Pyrex, quartz, diamond-like carbon, silicon, polydimethylsiloxane, biocompatible silicone based polymer, biocompatible metal or polymer;
wherein said biodegradable material is a polymer, polylactic acid (PLA), polyglycolic acid (PGA), copolymer of both poly (lacticglycolic) acid (PLGA), poly-β-hydroxybutyrate (PHB), polyanhydride, polyorthoester, polycaprolactone, polycarbonate, polyfumarate or polymethylmethacrylate; and
wherein said biodegradable hydrogel is collagen, collagen-glycosaminoglycan (GAG) copolymer, Type I collagen-κ elastin, alginate, calcium alginate, hyaluronan, methacrylated alginate, hyaluronan or agarose.

8. The device of claim 7 wherein said containment layer is surface modified with a mechanical, physical or chemical modification wherein said modification comprises a formation of channels, canals, ducts, conduits, tubes and utilizes masking techniques, plasma surface treatments, biological coating using polysaccharides, proteins, peptides, polymers, or glycoproteins, covalent linkages of peptides, integrins, nucleic acid, saccharides, lipopolysaccharides or amino acids, micromachining, wet or dry lithographic etching, plasma fluorine-based plasma etching, reactive ion etching, sputtering, plating, chemical and vapor deposition, physical vapor deposition and photoresist.

9. The device of claim 8 wherein said wells have a size from about 1 micron to about 5 millimeters, a depth from about 0.1 microns to 1 millimeter, and have a square, rectangular, oval, round or triangular shape and wherein a distance between the wells is from about 0.1 microns to about 3 millimeters.

10. The device of claim 9 wherein said wells are coated with an adhesive material selected from the group consisting of a protein, peptide, fibronectin, vitronectin, growth factor, poly-D-lysine or a peptide containing specific cell recognition sequences or with repulsive material selected from the group consisting of octodecyltrichlorosilane, polyethyleneglycol of molecular weight from 5,000 to 100,000, albumine or serum proteins.

11. The device of claim 10 wherein the microfluidic channel is a canal, conduit or tube of a diameter from about 1 to about 200 microns and a length of up to about 5 mm, wherein said channel connects the wells positioned on the same or different containment layers and permits the flow of cellular metabolites, hormones, proteins, water, nutrients, media or other solutions between the wells and layers.

12. The device of claim 11 wherein said microfluidic channels are equipped to establish, maintain and control a chemical, electrical or pressure potential, optical wave guide or ultrasound between the wells or between the support layer and the containment layer or between the support layer and the wells, or wherein said microfluidic channels permit build-up of osmotic gradient from the inlet port to the outlet port.

13. The device of claim 12 wherein said masking layer is removable.

14. The device of claim 13 wherein the removable masking layer is used for deposition of cells into the wells within the containment layers.

15. The device of claim 14 wherein the material used for fabrication of the masking layer is a glass, Pyrex, quartz, diamond-like carbon, silicon, polydimethylsiloxane, biocompatible silicone based polymer, biocompatible metal or polymer;
wherein said biodegradable material is a polymer, polylactic acid (PLA), polyglycolic acid (PGA), copolymer of both poly (lacticglycolic) acid (PLGA), poly-β-hydroxybutyrate (PHB), polyanhydride, polyorthoester, polycaprolactone, polycarbonate, polyfumarate or polymethylmethacrylate; and
wherein said biodegradable hydrogel is collagen, collagen-glycosaminoglycan (GAG) copolymer, Type I collagen-κ elastin, alginate, calcium alginate, hyaluronan, methacrylated alginate, hyaluronan or agarose.

16. The device of claim 15 wherein said masking layer comprises a multiplicity of openings, holes, perforations or slits distributed within said masking layer in a masking pattern corresponding to a pattern in which the wells are distributed within a containment layer into which the cells are deposited.

17. The device of claim 16 wherein for the cell deposition the openings on the masking layer are positioned directly above the well of the containment layer.

18. The device of claim 17 wherein said multiple containment layers with deposited cells within the wells are assembled and enclosed within the support layer cavity.

19. A method for the functional interaction and regulation of the same or different types of cells within a three-dimensional implantable device or within a stand-alone bioreactor or device, said method comprising steps:
a) fabricating a support layer, a multiplicity of containment layers comprising wells and one or several masking layers;
b) depositing the same or different types of cells within the wells of the same or different containment layer of said device using the masking layers;
c) assembling the containment layers of step b) into a three-dimensional structure within the support layer thereby generating a three-dimensional device;
d) introducing a buffer, saline, water, blood, serum or medium into the microfluidic channels interconnecting the individual wells through microfluidic channels; and
e) determining functionality of cells by measuring levels and production of cytokines, chemokines, growth factors, hormones, enzymes, metabolites, catabolites, proteins, peptides, nucleic acid, carbohydrates or lipids, or a combination thereof.

20. The method of claim 19 wherein the support layer is fabricated of glass, silicon, hardened polymer, titanium grade screen, film or porous material having a smooth or etched surface, Pyrex, quartz, diamond-like carbon, silicon, polydimethylsiloxane, biocompatible silicone based polymer, biocompatible metal or polymer;
wherein said support layer additionally optionally comprises a gated or nongated inlet port and a gated or nongated outlet port; and
wherein said support layer is flat or have rising edges or a rim to form a containment structure comprising a containment cavity for emplacement of one or several containment layers within said containment cavity.

21. The method of claim 20 wherein said containment layer is fabricated from a biocompatible biodegradable material, a biodegradable hydrogel or a rigid nonbiodegradable material selected from the group consisting of a glass, Pyrex, quartz, silicon, polydimethylsiloxane, biocompatible silicone based polymer, biocompatible metal, biocompatible polymer, polylactic acid (PLA), polyglycolic acid (PGA), copolymer of bothpoly (lacticglycolic) acid (PLGA), poly-β-hydroxybutyrate (PHB), polyanhydride, polyorthoester, polycaprolactone, polycarbonate, polyfumarate, polymethylmethacrylate, Type II collagen-glycosaminoglycan (GAG) copolymer, Type I collagen-κ elastin, alginate, calcium alginate, hyaluronan, methacrylated variation of alginates, hyaluronan and agarose.

22. The method of claim 21 wherein said containment layer is further surface modified with a mechanical, physical or chemical modification wherein said modification comprises a formation of channels, canals, ducts, conduits, tubes, utilizing masking techniques, plasma surface treatments, biological coating using polysaccharides, proteins, peptides, polymers, glycoproteins, covalent linkages of peptides, integrins, nucleic acid, saccharides, lipopolysaccharides or amino acids, micromachining, wet or dry lithographic etching, plasma fluorine-based plasma etching, reactive ion etching, sputtering, plating, chemical and vapor deposition, physical vapor deposition and photoresist.

23. The method of claim 22 wherein said wells positioned on said containment layer have a size from about 1 micron to about 5 millimeters and a depth from about 0.1 microns to about 1 millimeter, wherein said wells have a square, rectangular, oval, round or triangular shape and wherein a distance between the wells is from about 0.1 microns to about 3 millimeters.

24. The method of claim 23 wherein said wells are coated with a cell-specific adhesive or repulsive material selected from the group consisting of protein, peptide, fibronectin, vitronectin, growth factor, poly-D-lysine or a peptide containing specific cell recognition sequences permitting adhesion of cells to the wells during the cell deposition step.

25. The method of claim 24 wherein the microfluidic channel is a canal, conduit or tube of a diameter from about 1 to about 200 microns and length of up to about 5 mm, wherein said channel connects the wells positioned on different containment layers and permits the flow of cellular metabolites, catabolites hormones, proteins, peptides or nutrients secreted by the cells deposited within the wells.

26. The method of claim 25 wherein said microfluidic channels are equipped to establish, maintain and control a chemical, electrical or pressure potential or optical wave guide or ultrasound between wells or between the support layer and containment layer or between the support layer and wells or wherein said microfluidic channels permit build-up of osmotic gradient from the inlet port to the outlet port.

27. The method of claim 26 wherein the cell are deposited into the wells through a removable masking layer which is removed following the cell deposition through masking layers openings.

28. The method of claim 27 wherein said masking layer comprises a multiplicity of openings, holes, perforations or slits distributed within said masking layer in a masking pattern corresponding to a pattern in which the wells are distributed within the containment layer immediately below the masking layer into which the cells are deposited.

29. The method of claim 28 wherein for the cell deposition the openings on the masking layer are positioned directly above the well of the containment layer and wherein said cells are deposited in the wells directly under pressure, by centrifugation, suction, spraying, inkjet printing, spin-on in a centrifuge, electrical gradient or wherein the cells are deposited by injecting the appropriate microfluidic channel with a cell suspension under pressure, centrifugation, suction, spraying, inkjet printing, spin-on in a centrifuge or by electrical gradient.

30. The method of claim 29 wherein said multiple containment layers with deposited cells within the wells are enclosed within the containment cavity and products of cell metabolism or cell-cell interaction are removed through the outlet port and tested.

31. A process for fabricating a three-dimensional device comprises steps of:
a) fabricating a support layer;
b) fabricating a containment layer;
c) fabricating a masking layer;
d) depositing cells on the containment layer; and
e) assembling the three-dimensional device.

32. The process of claim 31 wherein in step a) said support layer is flat or have rising edges or a rim to form a containment structure comprising a containment cavity for emplacement of one or several containment layers within thus formed cavity and wherein said support layer is fabricated from a glass, silicon, hardened polymer, titanium grade screen, Pyrex, quartz, diamond-like carbon, silicon, polydimethylsiloxane, biocompatible silicone based polymer, biocompatible metal or polymer, film or porous material having a smooth or etched surface.

33. The process of claim 32 wherein the step a) further comprises attachment of a gated or nongated inlet port and a gated or nongated outlet port to said support layer, wherein said port is attached to the top, to the bottom or to the side of the support layer.

34. The process of claim 33 wherein the step b comprises fabrication of one or more containment layers comprising multiplicity of wells interconnected horizontally with microfluidic channels within one containment layer or vertically or with inclined microfluidic channels with other containment layers or with the support layer.

35. The process of claim 34 comprising fabrication of individual containment layers each comprised of wells coated with an adhesive material permitting a deposition of the same or different type of cells within wells of each containment layer.

36. The process of claim 35 wherein the containment layer is fabricated from a biocompatible biodegradable material, a biodegradable hydrogel, or a rigid nonbiodegradable material.

37. The process of claim 36 wherein said nonbiodegradable material is a glass, Pyrex, quartz, silicon, polydimethylsiloxane, biocompatible silicone based polymer, nonbiodegradable biocompatible metal or polymer wafer, and wherein said biodegradable material is a polymer, polyactic acid (PLA), polyglycolic acid (PGA), copolymer of both poly (lacticglycolic) acid (PLGA), poly-β-hydroxybutyrate (PHB), polyanhydride, polyorthoester, polycaprolactone, polycarbonate, polyfumarate or polymethylmethacrylate wafer and wherein said biodegradable hydrogel is collagen, collagenglycosaminoglycan (GAG) copolymer, Type I collagen-κ elastin, alginate, calcium alginate, hyaluronan, methacrylated variation of alginates, hyaluronan or agarose.

38. The process of claim 37 further comprising a surface modification of the containment layer with a mechanical, physical or chemical modification which modification comprises a formation of channels, canals, ducts, conduits, tubes, utilizing masking techniques, plasma surface treatments, biological coating using polysaccharides, proteins, peptides, polymers, or glycoproteins, covalent linkages of peptides, integrins, nucleic acid, saccharides, lipopolysaccharides, or amino acids, micromachining, wet or dry lithographic etching, plasma fluorine-based plasma etching, reactive ion etching, sputtering, plating, chemical and vapor deposition, physical vapor deposition and photoresist.

39. The process of claim 38 wherein said surface modification of said containment layer comprises formation of wells having sizes from about 1 micron to about 5 millimeters and depth from about 1 millimeter to 0.1 microns and having a square, rectangular, oval, round or triangle shape wherein a distance between the wells is from about 0.1 microns to about 3 millimeters.

40. The process of claim 39 wherein in step b) said wells are coated with an adhesive material selected from protein, peptide, fibronectin, vitronectin, growth factor, poly-D-lysine or a peptide containing specific cell recognition sequences to provide adhesion of the cells to the wells.

41. The process of claim 40 further including a formation of the microfluidic channels wherein a channel is a canal, conduit or tube of about 1 to about 200 microns diameter and up to about 5 mm length, wherein said channel connects the wells positioned on different containment layers and permits the flow of cellular metabolites, hormones, proteins, peptides or nutrients.

42. The process of claim 41 wherein said microfluidic channel is fabricated to be equipped with an entity permitting establishment, maintenance and control of chemical, electrical or pressure potential between the individual wells or between the support layer and the containment layer or between the support layer and the wells or where said microfluidic channel permits build-up of osmotic gradient from the inlet port to the outlet port.

43. The process of claim 42 wherein step c) comprises fabrication of one or more masking layers for cell deposition into the wells of the containment layers, said masking layers comprising openings having a pattern corresponding to several or all wells positioned on the containment layer immediately below the masking layer.

44. The process of claim 43 wherein said masking layers are fabricated for a permanent or removable use.

45. The process of claim 44 wherein the removable masking layer is fabricated of material which is removable by peeling it off the containment layer after the deposition of cells into the wells within the containment layer.

46. The process of claim 45 wherein the material used for fabrication of the masking layer is a glass, Pyrex, quartz, diamond-like carbon, silicon, polydimethylsiloxane, biocompatible silicone based polymer, biocompatible metal or polymer;
wherein said biodegradable material is a polymer, polylactic acid (PLA), polyglycolic acid (PGA), copolymer of both poly (lacticglycolic) acid (PLGA), poly-β-hydroxybutyrate (PHB), polyanhydride, polyorthoester, polycaprolactone, polycarbonate, polyfumarate or polymethylmethacrylate; and
wherein said biodegradable hydrogel is collagen, collagen-glycosaminoglycan (GAG) copolymer, Type I collagen-κ elastin, alginate, calcium alginate, hyaluronan, methacrylated alginate, hyaluronan or agarose.

47. The process of claim 46 wherein said masking layer comprises a multiplicity of openings, holes, perforations or slits distributed within said masking layer in a masking pattern corresponding to a pattern in which the wells are distributed within the containment layer into which the cells are deposited.

48. The process of claim 47 wherein for the cell deposition the openings on the masking layer are positioned directly above the well of the containment layer.

49. The process of claim 48 comprising, in step d), assembling of multiple containment layers with deposited cells within the wells into the three-dimensional device and wherein the wells and containment layers are interconnected through the microfluidic channels.

50. An implantable three-dimensional device fabricated from biocompatible or biodegradable material, or a combination of both, said device comprising a multiplicity of containment layers, said layers comprising a multiplicity of wells seeded with cells, cell clusters or tissue, said wells further interconnected vertically, horizontally or inclined through microchannels filled with a medium, buffer, water, blood, serum or other solution, said containment layers enclosed within a support layer comprising an inlet and an outlet, or both.

51. The device of claim 50 suitable for temporary or permanent replacement or tissue or organs.

52. A three-dimensional bioreactor device for production of cellular products, said bioreactor fabricated from biocompatible or biodegradable material, or a combination of both, said bioreactor device comprising a multiplicity of containment layers, said layer comprising a multiplicity of wells seeded with at lest two different types of cells, cell clusters or tissue, said wells interconnected vertically, horizontally or in incline through microchannels filled with a medium, saline, buffer, water, blood, serum or other solution, said containment layers enclosed within a support layer comprising an inlet and an outlet, or both.

53. The bioreactor of claim 52 wherein the cellular products are products of cell metabolism, proteins or hormones.
